(19) United States
(12) Patent Application Publication
(10) Pub. No.: US 2023/0151330 A1
(43) Pub. Date: May 18, 2023

(54) SYSTEMS AND METHODS FOR CELL CONVERSION

(71) Applicant: HigherSteaks Limited, Cambridge (GB)

(72) Inventors: Ruth Faram, Cambridge (GB); Benjamina Bollag, London (GB); Joy Emawodia, Bristol (GB); Stephanie Wallis, Bristol (GB)

(21) Appl. No.: 18/062,471

(22) Filed: Dec. 6, 2022

Related U.S. Application Data
(63) Continuation of application No. PCT/GB2021/051437, filed on Jun. 9, 2021.

(30) Foreign Application Priority Data
Jun. 10, 2020 (GB) 2008821.7

(51) Int. Cl. C12N 5/077; A23L 13/00

(52) U.S. Cl. CPC C12N 5/0658; A23L 13/00; C12N 2501/65; C12N 2513/00; C12N 2506/45; C12N 2533/00

(57) ABSTRACT

The present disclosure provides methods and systems for the large-scale generation of differentiated stem cells. The present disclosure is also directed to systems and methods for expanding and differentiating stem cells in large-scale culture using a bioreactor chamber.

### CROSS-REFERENCE

The present application is a continuation application of International Patent Application No. PCT/GB2021/051437, filed Jun. 9, 2021, which claims priority to United Kingdom Patent Application No. 2008821.7, filed Jun. 10, 2020, each of which is herein incorporated by reference in its entirety.

### BACKGROUND

The global population is expected to surpass 9 billion by 2050. While food production may need to substantially increase to fulfill the demand of the growing population, constraints on resources and arable land render many forms of food production infeasible for meeting this demand. Rapidly developing countries such as China, India, and Russia may increase consumption of richer food products, such as meat or other animal products (e.g. dairy, eggs) leading to an increased global demand on these items. According to the report of the Food and Agriculture Organization of the United Nations, the livestock sector is responsible for 18% of Greenhouse Gas (GHG) emissions, uses 30% of earth's terrain, 70% of arable land, and 8% of global freshwater. In addition, the world's demand for meat is expected to double by 2050, rendering traditional meat production systems unsustainable. Compared to several meat sources, particularly beef production, cultured meat may decrease 7-45% of energy use, 78-96% of the GHG emissions, 99% of land use and 82-96% of water use.

### SUMMARY

Cultured meat products can be an emerging technology in which animal muscle cells may be produced through in-vitro tissue culture in contrast to inefficient traditional livestock agriculture. Multiple cell types may be desirable in creating a cultured meat product, as traditional meat products generally do not solely consist of muscle-derived tissue, but fat, and connective tissue among others. Stem cell differentiation may provide an efficient avenue in producing multiple cell and tissue types for a heterogeneous cultured meat product. Forced, transient gene expression in cells such as stem cells and with simultaneous conditioning and expansion in a bioreactor may result in an efficient and holistic approach in developing a cultured meat product. Provided herein are methods and systems for producing edible meat product.

Various aspects of the present disclosure provide a method for differentiating or transdifferentiating cells to produce an edible meat product, the method comprising: delivering nucleic acid molecules comprising one or more ribonucleic acid (RNA) molecules into said cells; modulating gene expression of said cells with aid of said nucleic acid molecules or expression products thereof, to differentiate or transdifferentiate at least a subset of said cells to generate one or more target cells following delivery of said nucleic acid molecules, wherein upon said modulating, said nucleic acid molecules are not integrated into a genome of said cells; and producing said edible meat product using at least partially said one or more target cells generated in (b).

In some embodiments, said nucleic acid molecules comprise two or more different RNA molecules. In some embodiments, said cells comprise animal cells. In some embodiments, said animal cells comprise porcine cells.

In some embodiments, (c) comprises producing a tissue from said one or more target cells. In some embodiments, said tissue comprises muscle tissue, fat tissue, neural tissue, vascular tissue, epithelial tissue, connective tissue, bone or a combination thereof. In some embodiments, said one or more target cells comprise at least two different types of cells. In some embodiments, the method further comprises co-culturing said at least two types of target cells to generate a three-dimensional tissue. In some embodiments, said one or more target cells comprise muscle cells, fat cells, somite cells, neural cells, endothelial cells, smooth muscle cells, bone cells, or a combination thereof.

In some embodiments, said RNA molecules comprise MYOD1, MYOG, MYF5, MYF6, PAX3, or PAX7, or any combination or variant thereof. In some embodiments, said nucleic acid molecules comprise unlocked nucleic acid molecules. In some embodiments, at least one of said RNA molecules is modified with unlocked nucleic acid monomers (uRNAs). In some embodiments, said uRNAs are incorporated at various points along said at least one of said RNA molecules. In some embodiments, at least one of said RNA molecules is chemically modified to improve its stability. In some embodiments, chemical modifications to said at least one of said RNA molecules comprise anti-reverse cap analogues, 3′-globin UTR, poly-A tail modifications, or any combination thereof. In some embodiments, said RNA molecules comprise messenger RNA (mRNA), microRNA (miRNA), transfer RNA (tRNA), silencing RNA (siRNA), or a combination thereof.

The method of claim 16, wherein said nucleic acid molecules further comprise complementary deoxyribonucleic acid (cDNA) molecules. In some embodiments, said nucleic acid molecules are synthetic nucleic acid molecules. In some embodiments, said nucleic acid molecules are delivered to said cells with neutral or anionic liposomes, cationic liposomes, lipid nanoparticles, ionizable lipids, or any combination or variation thereof.

In some embodiments, said nucleic acid molecules are delivered in a single dose to said cells. In some embodiments, said nucleic acid molecules are delivered in at least two doses to said cells. In some embodiments, individual doses of said at least two doses are delivered at least 3 days apart. In some embodiments, individual doses of said at least two doses comprise different nucleic acid molecules. In some embodiments, said nucleic acid molecules are delivered at a concentration of at most 1 μM. In some embodiments, said nucleic acid molecules comprise siRNA targeting POUF51 (OCT3/4), KLF4, SOX2, or any combination or variant thereof. In some embodiments, said cells comprise stem cells, mature cells, or a combination thereof.

Various aspects of the present disclosure provide a method of generating an edible meat product from cells, comprising: bringing said cells in contact with a scaffold; subjecting at least a subset of said cells to a differentiation or a transdifferentiation process in the presence of said scaffold and with the use of a growth factor or a nucleic acid molecule, to thereby generate a tissue; and producing said edible meat product using said tissue.

In some embodiments, said scaffold is degradable. In some embodiments, said edible meat product comprises at least a portion of said scaffold. In some embodiments, said scaffold degrades at a rate of at least 1% per day during (b). In some embodiments, said cells comprise stem cells or mature cells. In some embodiments, comprising culturing said cells. In some embodiments, the method further comprises subjecting said cells to one or more expansion processes to expand said cells.

In some embodiments, said scaffold is configured to facilitate cell expansion during said one or more expansion processes in a bioreactor chamber. In some embodiments, (b) comprises generating differentiated or transdifferentiated cells from said cells, and optionally fusion of said differentiated or transdifferentiated cells within said scaffold. In some embodiments, (a) comprises depositing at least a subset of said cells on a surface of the scaffold. In some embodiments, said surface is an adherent surface.

In some embodiments, the method further comprises releasing cells of said at least said subset of said cells from said scaffold, and depositing said released cells on a surface of a separate scaffold. In some embodiments, said releasing is prior to (c). In some embodiments, at least 50% of fusion of said differentiated or transdifferentiated cells occurs prior to said releasing.

In some embodiments, said culturing is conducted in the presence of said scaffold. In some embodiments, said one or more expansion processes is conducted in the presence of said scaffold. In some embodiments, said culturing and said one or more expansion processes are performed in a same bioreactor chamber. In some embodiments, said culturing is performed in a bioreactor chamber and said one or more expansion processes are performed in an additional bioreactor chamber. In some embodiments, said additional bioreactor chamber comprises a plurality of additional bioreactor chambers each configured to facilitate an individual cell expansion process. In some embodiments, the method further comprises directing at least a subset of cultured cells from said bioreactor chamber to said plurality of additional bioreactor chambers to perform a plurality of expansion processes. In some embodiments, expansion processes of said plurality of expansion processes are performed sequentially, simultaneously, or a combination thereof. In some embodiments, said plurality of additional bioreactor chambers comprises at least two bioreactor chambers. In some embodiments, the method further comprises directing a medium through said bioreactor chamber and said additional bioreactor chamber to facilitate said culturing or said one or more expansion processes. In some embodiments, said medium is under continuous laminar flow. In some embodiments, said medium is configured to promote cell culturing or expansion processes. In some embodiments, the method further comprises directing said medium out of said additional bioreactor chamber. In some embodiments, the method further comprises filtering said medium directed out of said additional bioreactor chamber to remove undesired components from said medium, thereby generating a filtered medium. In some embodiments, the method further comprises recycling said filtered medium into said bioreactor chamber.

In some embodiments, said cells comprise animal derived stem cells. In some embodiments, said cells comprise porcine cells. In some embodiments, said cells comprise pluripotent stem cells. In some embodiments, said cells comprise embryonic stem cells (ESCs). In some embodiments, said cells comprise reprogrammed stem cells. In some embodiments, said cells comprise induced pluripotent stem cells (iPSCs).

In some embodiments, said scaffold comprises a polymeric material. In some embodiments, said polymeric material comprises a synthetic polymeric material. In some embodiments, said synthetic polymeric material comprises a polyethylene glycol biomaterial. In some embodiments, said polyethylene glycol biomaterial comprises an arginylglycylaspartic (RGD) motif. In some embodiments, said scaffold comprises a gellan gum biomaterial, a cassava biomaterial, a maize biomaterial, an alginate biomaterial, a corn-starch biomaterial, or any combination or variant thereof. In some embodiments, said method is performed in vitro.

In some embodiments, said edible meat product is in a unit form of at least 50 grams. In some embodiments, said edible meat product is in a solid state with a texture comparable with that of an in-vivo derived steak including loins. In some embodiments, said edible meat product is in a solid state with a texture comparable with that of an in-vivo derived bacon. In some embodiments, said edible meat product is in a solid state with a texture comparable with that of an in-vivo derived pork belly. In some embodiments, said edible meat product is in a solid state with a texture comparable with that of an in-vivo derived mince. In some embodiments, said edible meat product is in a solid state with a texture comparable with that of an in-vivo derived sausage. In some embodiments, said edible meat product is in a solid state with a texture comparable with that of an in-vivo derived ribs. In some embodiments, said edible meat product is in a solid state with a texture comparable with that of an in-vivo derived chops. In some embodiments, said edible meat product is in a solid state with a texture comparable with that of an in-vivo derived cured meat product. In some embodiments, said edible meat product is incorporated into a further processed food product. In some embodiments, said edible meat product comprises nutritional additives comprising vitamins and minerals.

In some embodiments, said one or more expansion processes comprise passaging at least a subset of cultured cells. In some embodiments, said passaging comprises passing an enzyme over said at least said subset of said cultured cells to detach said cells from a surface of said scaffold.

Various aspects of the present disclosure provide a method for generating an edible meat product from cells, the method comprising: modulating expression of one or more genes in said cells in a transient and non-integrative manner using two or more ectopic differentiation factors to generate progenitor cells; differentiating at least a subset of said progenitor cells to generate terminally differentiated cells; and producing said edible meat product based at least partially on said terminally differentiated cells.

In some embodiments, the method further comprises subjecting one or more of said cells, said progenitor cells, and said terminally differentiated cells to a culturing and/or an expansion process. In some embodiments, said culturing and said expansion processes are performed in a same, or different bioreactor chambers. In some embodiments, said terminally differentiated cells comprise muscle cells, fat cells, somite cells, neural cells, endothelial cells, smooth muscle cells, bone cells, or a combination thereof. In some embodiments, said ectopic differentiation factors comprise nucleic acids, polypeptides, small molecules, growth factors, or any combination thereof. In some embodiments, (b) comprises differentiating said progenitor cells by arresting the cell cycle of cells.

In some embodiments, said ectopic differentiation factors arrest the cell cycle of cells through reducing or removing growth factors from said cells. In some embodiments, said growth factors comprise LIF, FGF, BMP, activin, MAPK, TGF-β, or any combination thereof. In some embodiments, said arresting the cell cycle of cells occurs by reducing or removing serum levels in a solution in which cell culturing is conducted.

Various aspects of the present disclosure provide a method for generating an edible meat product using cells, the method comprising: delivering into said cells two or more different types of nucleic acid molecules comprising messenger ribonucleic acid (mRNA), microRNA (miRNA), transfer RNA (tRNA), silencing RNA (siRNA), or complementary deoxyribonucleic acid (cDNA);

modulating gene expression of said cells with aid of said two or more different types of nucleic acid molecules or expression products thereof, to generate one or more target cells following delivery of said two or more different types of nucleic acid molecules, wherein said modulating is in a transient manner such that said nucleic acid molecules are not integrated into a genome of said cells; producing said edible meat product using at least partially said one or more target cells generated in (b).

In some embodiments, said two or more different types of nucleic acid molecules are generated by an in vitro process. In some embodiments, said two or more different types of nucleic acid molecules comprise mRNA and siRNA. In some embodiments, said mRNA comprises MYOD1, MYOG, MYF5, MYF6, PAX3, PAX7, or any combination or variant thereof. In some embodiments, said siRNA targets POUF51 (OCT3/4), KLF4, SOX2, or any combination or variant thereof. In some embodiments, said two or more different types of nucleic acid molecules comprise cDNA and siRNA. In some embodiments, said cDNA comprises MYOD1, MYOG, MYF5, MYF6, PAX3, PAX7, or any combination or variant thereof.

In some embodiments, (b) comprises enhancing, reducing, or inhibiting said gene expression. In some embodiments, said gene expression comprises expression of one or more genes in said cells. In some embodiments, (b) comprises enhancing expression of a first gene of said one or more genes, and inhibiting expression of a second gene of said one or more genes.

In some embodiments, said delivering comprises a single dose of said two or more different types of nucleic acid molecules. In some embodiments, said delivering comprises at least two doses of said two or more different types of nucleic acid molecules. In some embodiments, individual doses of said at least two doses comprises different nucleic acid molecules. In some embodiments, said at least two doses comprise different concentrations of said two or more different types of nucleic acid molecules.

Various aspects of the present disclosure provide an edible meat product prepared by a process comprising the steps of: bringing a plurality of cells in contact with a scaffold; subjecting at least a subset of said plurality of cells to a differentiation or a transdifferentiation process in the presence of said scaffold and with the use of a growth factor or a nucleic acid molecule, to thereby generate a tissue; and producing said edible meat product using said tissue. In some embodiments, said tissue comprises at least two types of cells. In some embodiments, said at least two types of cells comprise myocytes and adipocytes. In some embodiments, a ratio of said myocytes to said adipocytes is between 99:1 and 80:20. In some embodiments, said edible meat product comprises at least 2% by mass of said scaffold. In some embodiments, said edible meat product comprises less than 5% of muscle extracellular matrix by mass. In some embodiments, said plurality of cells comprise stem cells or mature cells. In some embodiments, said process further comprises culturing at least a subset of said plurality of cells. In some embodiments, said process further comprises subjecting at least a subset of said plurality of cells to one or more expansion process. In some embodiments, said scaffold comprises an extended 3-dimensional structure. In some embodiments, (b) comprises generating differentiated or transdifferentiated cells from said cells, and optionally fusion of said differentiated or transdifferentiated cells within said scaffold.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a computer system that is programmed or otherwise configured to implement methods provided herein.

FIG. 2 illustrates an example flow chart schematic wherein an edible biomaterial scaffold and species-specific constructs may be produced, the cells may be expanded in one or a plurality of bioreactors in contact with the scaffolds and constructs, differentiated in one or a plurality of bioreactors, and laminar media flowed and recycled between bioreactor tanks.

FIG. 3A illustrates an example of the formation of multinucleated MYOD1 expressing muscle fibers 10 days after differentiation with MYOD mRNA. FIG. 3B illustrates an example of the formation of multinucleated, aligned MYOD1 expressing muscle fibers 30 days after differentiation with MYOD mRNA.

FIG. 4 illustrates a schematic demonstrating an example bioreactor system for use in accordance with an example of the present disclosure.

FIG. 5A illustrates a schematic demonstrating an example composition of shelves in a bioreactor. Each shelf is shown in blue. Media is shown in pink and the flow of media with arrows. A thin yellow layer between the media and shelf is shown, indicating the cell surface coating. Cells are grown on top of the cell surface coating and media flows over them. FIG. 5B illustrates the direction of flow of media (arrows) throughout each bioreactor and orientation of the shelves (horizontal lines).

FIG. 6A-C illustrate three examples of multinucleated muscle fibers 14 days after differentiation with porcine-specific MYOD1 mRNA. FIG. 6A provides a phase contrast image of the muscle fibers. FIG. 6B provides fluorescence image of the muscle fibers with contrasting phalloidin actin, MYOD1, and DAPI nuclear stains. FIG. 6C provides a fluorescence image of the muscle fibers with contrasting myosin heavy chain and DAPI nuclear stains.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The use of the word “a” or “an,” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” is used to indicate that a value includes the inherent variation of error for the device, the method being employed to determine the value, or the variation that exists among the study subjects. Unless otherwise specified based upon the above values, the term “about” means±5% of the listed value.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “flavor,” as used herein, generally refers to the taste and/or the aroma of a food or drink.

The term “food product,” as used herein, generally refers to a composition that can be ingested by humans or animals, including e.g., domesticated animals (e.g., dogs, cats), farm animals (e.g., cows, pigs, horses), and wild animals (e.g., non-domesticated predatory animals). The term may refer to any substance that can be used or prepared for use as food, such as any substance that can be metabolized by a human or animal to give energy and build tissue. It may be eaten or drunk by any human or animal for nutrition or pleasure. A food product may comprise carbohydrates, fats, proteins, water, or other ingredients which can be ingested by humans or animals.

As used herein, the term “nucleic acid” generally refers to a polymeric form of nucleotides of various lengths (e.g., at least 2, 3, 4, 5, 6, 7, 8, 9, 10, 100, 500, 1000 or more nucleotides), either deoxyribonucleotides or ribonucleotides, or analogs thereof. A nucleic acid may include one or more subunits selected from adenosine (A), cytosine (C), guanine (G), thymine (TO, and uracil (U), or variants thereof. A nucleotide can include any subunit that can be incorporated into a growing nucleic acid strand. Such subunit can be A, C, G, T, or U, or any other subunit that is specific to one of more complementary A, C, G, T, or U, or complementary to a purine (e.g., A or G, or variant thereof) or pyrimidine (e.g., C, T, or U, or variant thereof). In some examples, a nucleic acid may be single-stranded or double stranded, in some cases, a nucleic acid molecule is circular. Non-limiting examples of nucleic acids include deoxyribonucleic acid (DNA) and ribonucleic acid (RNA). Nucleic acids can include coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid molecule may comprise one or more modified nucleotides, such as methylated nucleotides and nucleotide analogs. A nucleic acid may be synthetic.

The Food and Agriculture Organization of the United Nations estimates the demand for meat may likely increase by more than two-thirds in the next 40 years with a booming global population and current production methods are not sustainable to meet this demand. Meat products are currently taken from the muscles of animals with butchers carving out corresponding cuts of livestock to be sold as steak, chicken breast, lamb chops, fish fillet, pork chops, etc. Meat products can also include meat-product derivatives such as ground meat that may be processed into meatball, hamburger patty, fishballs, sausage, salami, bologna, ham, etc. as well as seasoned or dried muscle tissues or meat such as jerky. Meat products using animals may be inefficient food sources with livestock consuming 70% of all wheat, corn, and other grain produced in the United States alone and over a thousand pounds of water needed to produce one pound of beef. Livestock is responsible for 18% of Green House Gas (GHG) emissions, uses 30% of Earth's terrain, 70% of arable land, and 8% of freshwater globally.

Factory farming and poor animal welfare conditions in livestock agriculture are a cause for foodborne illnesses, with harmful bacteria such as Salmonella, E. Coli, and Campylobacter inherent to raw meat. As many as 25% of broiler chickens and 45% of ground chickens may test positive for Salmonella and The Center for Disease Control estimates that Campylobacter infects 70% to 90% of all chickens. Multidrug resistance in bacteria is encouraged by industrial meat production with 70% of all antibiotics used in the United States given to farm animals as a food additive. Antibiotic overuse may be the primary cause of antibiotic resistant bacteria and bacteria resistant to colistin, a last-line therapy in treating Gram-negative infections, emerged in Chinese pig farms in 2016. Industrial livestock operations have long been a target of virologists in discovering novel zoonotic infections with the H1N1, H5N1, and H3N2 influenzas circulating widely in chicken and pig farms and the 2019-2020 SARS-CoV-2 pandemic potentially arising from wet market conditions. A more efficient, safer, and healthier method of meat production than current methods of production is needed.

Cultured meat may be an emerging technology in which animal originated cells (e.g., animal muscle cells) are produced in controlled in-vitro environments using tissue culture techniques in contrast to traditional livestock agriculture. Compared to current meat sources, cultured meat may decrease 7-45% of energy use, 78-96% of GHG emissions, 99% of land use, and 82-96% of water use. Meat produced in a sterile, controlled environment may improve food safety. Provided herein are systems and methods for producing a meat product for food consumption. An edible food product comprising a textured protein may be derived from the expansion and differentiation or trans-differentiation of cells. The cells may be animal cells. The animal cells may be non-human cells. The cells may comprise porcine cells. The cells may be stem cells or mature cells from which the differentiated or transdifferentiated cells may be generated. The method may be conducted with the aid of a scaffold in a bioreactor. The scaffold may be degradable and/or suitable for human consumption. Expansion may comprise growing a population of cells exponentially into larger systems. Cellular expansion may be a process that results in an increase of the number of cells and may be affected by the balance between cell divisions and cell loss through death or differentiation.

In some aspects, the present disclosure may provide systems and methods for producing tissue engineered food products. A food product may be any composition that can be ingested and metabolized by humans or animals to give energy and build tissue. It may be eaten or drunk by any human or animal for nutrition or pleasure. A food product may comprise carbohydrates, fats, proteins, water, or other ingredients. A food product may be combined with or added to other ingredients to make compositions that can be ingested by humans or animals. A food product may be a meat product. A meat product may encompass any animal flesh (e.g., beef, pork, poultry, fish) capable of use as human food. A meat product may be generated from different sources. For example, a meat product may be made wholly or in part from any meat or other portion of the carcass of any cattle, sheep, swine, goats or poultry. A meat product may be an animal flesh-like product, such as a cultured meat, that is eaten as food which has the organoleptic property of meat. A cultured meat may be a cultured food product which may have one or more properties of natural meat. A cultured meat product may comprise the in-vitro cell culture of animal cells such as muscle cells, fat cells, connective tissue, blood, or other components (e.g., proteins) to be used as a meat product. Cultured meat may include cultured animal cells. A cultured meat may comprise an intact, flesh-like composition with minimal processing or may comprise all any type of meat, poultry, or game products, in pieces, cuts, or comminuted, which may be processed to any degree or incorporated into a food product of heterogenous composition such as a nugget or a patty. A cultured meat may resemble a corresponding cut of beef, poultry, lamb, fish, pork, or other animal product. A cultured meat may resemble a whole-meat product such as a steak (including loins), mince, sausage, ribs, chops, cured meats, pork belly, bacons, chicken breast, lamb chops, fish fillet, or pork chops. A cultured meat may be a meat product or meat-product derivative prepared, for example, by grounding or shredding the muscle tissues grown in vitro and mixed with appropriate seasoning. Such a meat product may be processed into ground meat, meatball, hamburger patty, fishball, sausage, nugget, salami, bologna, ham, or lunchmeats. A meat product may also include a seasoned or dried product such as a jerky. A meat product may be used to generate any kind of food product originating from or similar to the meat of an animal. A meat product may comprise a hybrid food product comprising a plant-originated substance and a cultured meat, cells, or substances interconnected with the plant-originated substance to form a unified food product with an improved organoleptic and nutritional value compared with a sole plant-originated substance. A meat product may be free of bodily fluids e.g., saliva, serum, plasma, mucus, urine, feces, tears, milk etc. or may comprise a bodily fluid.

Cultured cells or tissues may be combined with at least one other ingredient. Cultured cells or tissues may be combined with at least one other ingredient to obtain a food product having a desired texture, moisture retention, product adhesion, or any combination thereof. A cultured cell may be a cell grown under controlled conditions such as an in-vitro condition outside their natural environment. An ingredient may comprise a binder, filler, or extender. A filler or binder may comprise a non-meat substance comprising carbohydrates such as a starch. Fillers and binders may include potato starch, flour, eggs, gelatin, carrageenan, and tapioca flour. An extender may have a high protein content. Extenders may comprise soy protein, milk protein, or meat-derived protein. Ingredients that provide flavor, texture, or other culinary properties may be added to a meat product. For example, extracellular matrix proteins may be used to modulate structural consistency and texture. Proteins such as heme or collagen may be incorporated into the extracellular matrix to contribute to the taste and texture of the final food product. Nutrients such as vitamins that are normally lacking in meat products from whole animals may be added to increase the nutritional value of the meat product. This may be achieved either through straight addition of the nutrients to a growth medium or by alternative methods. For example, the enzymes responsible for the biosynthesis of a particular vitamin, such as Vitamin D, A, or the different Vitamin B complexes, may be transfected into the cultured muscle cells to produce the particular vitamin within those cells.

A cultured meat product may be produced by culturing cells in-vitro into a tissue product. A cell may comprise a cell membrane, at least one chromosome, composed of genetic material, cytoplasm, and various organelles which are adapted or specialized to perform one or more vital functions, such as energy and proteins synthesis, respiration, digestion, storage and transportation of nutrients, locomotion, or cell division. A cell may comprise one or a plurality of cells. A cell may comprise a somatic cell, a terminally differentiated cell, a stem cell, a germ cell, a mature cell, or others alike. A somatic cell may be any cell forming the body of an organism that are not germline cells. Mutations in somatic cells may affect the individual organism but are not passed onto offspring. A cell may comprise satellite cells, myoblasts, myocytes, fibroblasts, hepatocytes, vascular endothelial cells, pericytes, extraembryonic cell lines, somatic cell lines, adipocytes, chondrocytes, somite cells, blood cells, mesenchymal cells, or stem cells. A myocyte may be the smallest subunit of all muscular tissues. Skeletal muscle myocytes may differentiate from mesenchymal stem cells to skeletal muscle myoblasts and fuse into multinucleated muscle fibers, myofibrils, that behave as a unit. These myofibrils may be composed of overlapping filaments, myofilaments, that are both thick and thin and allow for a contraction of its length using a series of motor proteins. An adipocyte may be a cell primarily composed of adipose tissue, specialized in synthesizing and storing energy as fat. Adipocytes may be derived from mesenchymal stem cells through adipogenesis. Adipocytes may be white adipocytes, which store energy as a single large lipid droplet and have important endocrine functions, and brown adipocytes which store energy in multiple small lipid droplets but specifically for use as fuel to generate body heat. Cells may be myogenic cells. Myogenic cells may be natively myogenic (e.g. are myogenic cells that are cultured in the cultivation infrastructure). Natively myogenic cells include, but are not limited to, myoblasts, myocytes, satellite cells, side population cells, muscle derived stem cells, mesenchymal stem cells, myogenic pericytes, or mesoangioblasts. Myogenic cells may not be natively myogenic (e.g. are non-myogenic cells that are specified to become myogenic cells in the cultivation infrastructure). Non-myogenic cells include embryonic stem cells, induced pluripotent stem cells, extraembryonic cell lines, and somatic cells other than muscle cells. A cell may be a wild-type cell or may be a genetically modified cell (e.g., transgenic, genome edited). Non-myogenic cells may be modified to become myogenic cells through the expression of one or more myogenic transcription factors such as MYOD1, MYOG, MYF5, MYF6, PAX3, PAX7, paralogs, orthologs, or genetic variants thereof. Myoblast determination protein (MYOD) may be a skeletal muscle specific transcription factor and protein in animals that play a significant role in regulating muscle differentiation. MYOD may commit mesoderm cells to a skeletal myoblast lineage and regulate that differentiation and proliferation of myoblasts. MYOD may be considered a master regulatory gene of skeletal muscle differentiation and its ability to convert fibroblasts and other cell types into skeletal muscle supports its central role in myogenesis.

A cell may differentiate into specific types of cells such as muscle cells including skeletal muscle cells or smooth muscle cells. Differentiation may refer to the process during which young, unspecialized cells take on individual characteristics and reach their specialized form and function. Cell differentiation may allow a single cell and genotype to result in tens to hundreds of different cell types and phenotypes. Through differentiation a totipotent cell may move through pluripotency or multipotency, eventually reaching a lineage committed state. A cell may comprise a stem cell which may be any unspecialized cell capable of renewing themselves through cell division which have the developmental potential to differentiate into multiple cell types. A stem cell may be any unspecialized cell capable of self-renewal through cell division which may have the developmental potential to differentiate into multiple cell types, without a specific implied meaning regarding developmental potential, for example a stem cell can be totipotent, pluripotent, multipotent, etc. A stem cell may be a cell capable of proliferation and giving rise to more such stem cells while maintaining its developmental potential. A stem cell may refer to any subset of cells that have the developmental potential, under particular circumstances, to differentiate to a more specialized or differentiated phenotype, and which retain the capacity, under certain circumstances, to proliferate without substantially differentiating. A stem cell may refer to a naturally occurring parent cell whose descendants (progeny cells) specialize, often in different directions, by differentiation, e.g., by acquiring completely individual characters, as occurs in progressive diversification of embryonic cells and tissues. Some differentiated cells may have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype.

A stem cell may be totipotent, pluripotent, multipotent, oligopotent, or unipotent. A stem cell may comprise an embryonic stem cell, animal stem cell, adult stem cell, induced pluripotent stem cell, reprogrammed stem cell, mesenchymal stem cell, hematopoietic stem cell, or a progenitor cell. An embryonic stem cell may refer to embryonic cells capable of differentiating into cells of all three embryonic germ layers (the endoderm, ectoderm and mesoderm), or remaining in an undifferentiated state. The embryonic stem cells may comprise cells which are obtained from the embryonic tissue formed after gestation (e.g., blastocyst) before implantation of the embryo, such as a pre-implantation blastocyst, extended blastocyst cells which are obtained from a post-implantation/pre-gastrulation stage blastocyst, embryonic germ cells which are obtained from the genital tissue of a fetus, and cells originating from an unfertilized ova which are stimulated by parthenogenesis (parthenotes). An embryonic stem cell has unlimited self-renewal ability and pluripotent differentiation ability. An adult stem cell may be any stem cell derived from a somatic tissue of either a postnatal or prenatal animal. An adult stem cell may be capable of indefinite self-renewal while maintaining its undifferentiated state and is multipotent, capable of differentiation into multiple cell types. Adult stem cells can be derived from any adult, neonatal or fetal tissue such as adipose tissue, skin, kidney, liver, prostate, pancreas, intestine, bone marrow and placenta. Induced pluripotent stem cells or iPSCs may comprise any cells obtained by de-differentiation of adult somatic cells which are endowed with pluripotency, a cell being capable of differentiating into the three embryonic germ cell layers, the endoderm, ectoderm and mesoderm. Such cells may be obtained from a differentiated tissue (e.g. a somatic tissue such as skin) and undergo de-differentiation by genetic manipulation which reprogram the cell to acquire stem cell-like characteristics. iPSCs may be formed through a process that reverses the developmental potential of a cell or population of cells (e.g., a somatic cell). An iPSC may be a cell that has undergone a process of driving a cell to a state with higher developmental potential, such as a cell that is driven backwards to a less differentiated state. The somatic cell, prior to induction to an iPSC, can be either partially or terminally differentiated. There may be a complete or partial reversion of the differentiation state, i.e., an increase in the developmental potential of a cell, to that of a cell having a pluripotent state. A somatic cell may be driven to a pluripotent state, such that the cell has the developmental potential of an embryonic stem cell, similar to an embryonic stem cell phenotype. Induction of a somatic cell may also encompass a partial reversion of the differentiation state or a partial increase of the developmental potential of a cell, such as a somatic cell or a unipotent cell, to a multipotent state. Induction may also encompass partial reversion of the differentiation state of a cell to a state that renders the cell more susceptible to complete induction to a pluripotent state when subjected to additional manipulations. A stem cell may comprise a reprogrammed cell. Cellular reprogramming may be a process that reverses the developmental potential of a cell or population of cells (e.g., a somatic cell). Reprogramming may be a process of driving a cell to a state with higher developmental potential, such as driving a cell backwards to a less differentiated state. The cell to be reprogrammed can be either partially or terminally differentiated prior to reprogramming. Reprogramming may infer a complete or partial reversion of the differentiation state, such as an increase in the developmental potential of a cell, to that of a cell having a pluripotent state, driving a somatic cell to a pluripotent state, such that the cell has the developmental potential of an embryonic stem cell, such as an embryonic stem cell phenotype, or may encompass a partial reversion of the differentiation state or a partial increase of the developmental potential of a cell, such as a somatic cell or a unipotent cell, to a multipotent state. Reprogramming may also encompass a partial reversion of the differentiation state of a cell to a state that renders the cell more susceptible to complete reprogramming to a pluripotent state when subjected to additional manipulations. Hematopoietic stem cells may be adult tissue stem cells, including stem cells obtained from blood or bone marrow tissue of an individual at any age or from cord blood of a newborn individual. These cells may give rise to other blood cells during hematopoiesis. Hematopoietic stem cells may have the ability to self-renew and may be pluripotent, able to generate any and all diverse mature functional hematopoietic cell types such as erythrocytes, platelets, basophils, neutrophils, eosinophils, monocytes, T-lymphocytes, and B-lymphocytes. Mesenchymal stem cells may be multipotent stromal cells that can differentiate into a variety of cell types, including osteoblasts (bone cells), chondrocytes (cartilage cells), myocytes (muscle cells), adipocytes (fat cells which give rise to marrow adipose tissue), and neuron-like cells. Mesenchymal stem cells may be derived from the marrow as well as other non-marrow tissues, such as placenta, umbilical cord blood, adipose tissue, adult muscle, corneal stroma or the dental pulp of deciduous baby teeth. The cells may not have the capacity to reconstitute an entire organ but may be capable of self-renewal while maintaining their multipotency. A progenitor cell may comprise any cell that maintains the ability to differentiate into at least one specific type of cells but is more specified than a stem cell and pushed to differentiate to a target cell. Progenitor cells may not be able to replicate indefinitely and may only divide a limited number of times. A cell may also comprise a reprogrammed cell such as a transdifferentiated mature cell wherein a somatic cell may be reprogrammed or otherwise induced into another lineage without going through an intermediary proliferative stem cell phase. Transdifferentiated mature cells may be somatic cells that are reprogrammed or otherwise induced into another lineage without going through an intermediate proliferative pluripotent stem cell stage. Direct transdifferentiation of mature cells may occur through transient, forced expression of transcription factors, different methods of transfection, culture conditions, and supplementation of small molecules or growth factors.

A cell may be derived from any non-human animals such as mammals (e.g. cattle, buffalo, pigs, sheep, deer, etc.), birds (e.g. chicken, ducks, ostrich, turkey, pheasant, etc.), fish (e.g. swordfish, salmon, tuna, sea bass, trout, catfish, etc.), invertebrates (e.g. lobster, crab, shrimp, clams, oysters, mussels, sea urchin, etc.), reptiles (e.g. snake, alligator, turtle, etc.), or amphibians (e.g. frog legs). A cell may be a mammalian cell. In some cases, a mammalian cell may be a bovine cell, a bubaline cell, a porcine cell, an ovine cell, a caprine cell, a cervine cell, a bisontine cell, a cameline cell, an elaphine cell, or a lapine cell. A cell may be a bird cell. In some cases, a bird cell may be an anatine cell, galline cell, an anserine cell, a meleagrine cell, a struthionine cell, or a phasianine cell. A cell may be a piscine cell. A cell may be an invertebrate cell. In some cases, an invertebrate cell may be a homarine cell, a cancrine cell, or an ostracine cell. A cell may be a reptile cell. In some cases, a reptile cell is a serpentine cell, an eusuchian cell, or a chelonian cell. A cell may be an amphibian cell. In some cases, an amphibian cell is a ranine cell.

A cell-derived meat product may comprise one cell type, such as a skeletal muscle myocyte, or a heterogeneous co-culture composition, such as a skeletal muscle myocyte and an adipocyte composition. A plurality of single cell types may be cultured individually and then combined into a final product. A meat product may be derived from muscle cells grown ex vivo and may include fat cells derived also from any non-human animals. A ratio of muscle cells to fat cells may be regulated to produce a meat product with optimal flavor and health effects. A meat product may be derived from myocytes, myoblasts, osteoblasts, osteoclasts, adipocytes, neurons, endothelial cells, smooth muscle cells, cardiomyocytes, fibroblasts, hepatocytes, chondrocytes, kidney cells, cardiomyocytes, or a combination thereof. The tissue may comprise a muscle tissue, fat tissue, neural tissue, vascular tissue, epithelial tissue, connective tissue, bone, or a combination thereof. A meat product may comprise an organ meat or connective tissue meat such as liver, kidney, heart, tongue, brain, trotters, tripe, sweetmeat, gizzard, caul, sweetbread, pancreas, stomach, lungs, intestine, placenta, chitterlings, testicles, or feet. Regulation may be achieved by controlling the ratio of muscle and fat cells that are initially seeded in culture and/or by varying, as desired, the concentrations and ratio of growth factors or differentiation factors (e.g. mRNA) or other elements that act upon the muscle cells, fat cells, or another cell type.

### Cell Differentiation

An aspect of the present disclosure provides a method of producing an edible meat product using animal cells (e.g., porcine cells). The method may be performed in-vitro. The method may comprise delivering nucleic acid molecules into the cells. The nucleic acid molecules may comprise one or more RNA molecules. Following the delivery, gene expression of the cells (e.g., expression of one or more genes in the cells) may be modulated by the nucleic acid molecules or expression products of the nucleic acid molecules (e.g., proteins). Upon the modulation, the cells may be differentiated or trans-differentiated into one or more target cells including e.g., progenitor cells, or terminally differentiated cells. The cell differentiation or trans-differentiation may be conducted in a transient manner, during which the nucleic acid molecules delivered into the cells are not integrated into a genome of the cells. Subsequent to generation of the target cells, the meat product may be produced using at least a portion of the target cells.

In some cases, the target cells are terminally differentiated cells which may be used to produce a tissue for producing edible meat product. A terminally differentiated cell may be a cell that in the course of acquiring specialized functions, and thus may not be able to transform into other types of cells. These cells may constitute most of the mammalian body and may be unable to proliferate. The terminally differentiated cells may comprise one type of terminally differentiated cells or may comprise at least two types of terminally differentiated cells. The two or more types of terminally differentiated cells may comprise myocytes, myoblasts, osteoblasts, osteoclasts, adipocytes, neurons, endothelial cells, smooth muscle cells, cardiomyocytes, fibroblasts, hepatocytes, or chondrocytes. The tissue may comprise a muscle tissue, fat tissue, neural tissue, vascular tissue, epithelial tissue, connective tissue, bone, or a combination thereof. A muscle tissue may be a form of striated muscle that provides vertebrates with locomotive ability as well as serving metabolic and endocrine roles. Skeletal muscle may be comprised of fused and oriented myoblasts which allows a large force to be generated during contraction enabling movement. The skeletal muscle mass of livestock, fish, and game used to produce human food may represent 35-60% of their bodyweight and exhibit a wide diversity in shape, size, anatomical location, and physiological function. Adipose tissue or fat tissue may be a loose connective tissue composed of adipocytes. The main function of adipose tissue may be to store energy in the form of fat. Adipose tissue may be intramuscular or extra muscular. Intramuscular fat content may affect the flavor, juiciness, tenderness, and visual characteristics of meat. There may be a general relationship between the role of increased intramuscular fat and palatability with respects to food products.

A cell phenotype or genotype may be determined using polymerase chain reaction (PCR), immunohistochemistry, or mass spectrometry. The mass spectra obtained different cells may provide a fine-grained description of the proteomic state of a cell culture or a fingerprint of the cell type which may be used to identify the differentiation states of cells. A determined proteomic fingerprint of cells may be used to characterize other compounds and pinpoint their effect on antibacterial drug targets. Mass spectra of cell cultures may require minimal sample preparation, small sample amounts, and provide a high-throughput method of identification for large scale cell cultures enabling rapid identification of cell types. Different desorption and ionization ability in matrix-assisted laser desorption/ionization mass spectrometry (MALDI MS), several pairs of peptides and proteins with similar molecular weight can be regarded as internal standards for each other, especially for those sharing similar structure. The relative intensity of peak pairs detected in the cell lines may be highly conserved. When different species of cells were mixed or co-cultured, the ratiometric peak information can be utilized as a cellular fingerprint for quantitative analysis thus enabling rapid identification and quantification of different cell types according to the ratio values of these peak pairs in mass spectra. Coupled with imaging technology, distribution and proportion of cell types in a whole tissue can be estimated enabling the ratio of different cell types in a heterogeneous tissue in a meat product.

In contrast to traditional livestock agriculture, cells having a self-renewal capacity may be isolated or created and grown in cell culture indefinitely into a tissue structure similar to meat. Such cells may be naturally capable of self-renewal such as embryonic stem cells and pluripotent progenitor cells or may be manipulated to acquire the ability to self-renew. Induced pluripotent stem cells (iPSCs) are artificially induced embryonic stem cell-like cells. These cells may be created by reprogramming somatic cells through the introduction of reprogramming factors (transcription factors that drive expression of pluripotency genes). iPSCS are self-replicating and may be expanded to increase the population. Desired cell types, such as skeletal muscle myocytes or adipocytes, may be generated from iPSCs using manipulation of the cell's environment and differentiation factors. Cultured cells may be directed down a differentiation pathway to generate a desired cell type such as into muscle cells, adipose cells, or organ cells. As traditional meat products are not a homogenous composition, rather a heterogeneous combination of multiple tissue and cell types, a population of cells may be differentiated into multiple cell types or independent cell populations may be differentiated into distinct cell types and subsequently combined to produce a composition comprising both muscle and fat cells, or other desired cell types.

Directed differentiation of cells may occur with chemical methods using differentiation factors and small molecules, genetic methods using gene editing techniques to force gene expression within the cells, or viral transduction where viral constructs encoding a gene insert of interest are used to infect and promote forced gene expression. Modulating the expression of one or more genes in a stem cell may comprise the introduction of RNA. “Expression,” “cell expression” or “gene expression” may refer to a process by which information from a gene can be used in the synthesis of a functional gene product. These products may be proteins or may be a functional RNA. Expression may comprise genes transcribed into mRNA and then translated into protein or genes transcribed into RNA but not translated into protein. The RNA introduced may comprise a myogenic gene such as MYOD1, MYOG, MYF5, MYF6, PAX3, PAX7, or any variants, analogs, or combinations thereof.

RNA may be introduced or delivered into a cell using an expression vector. A vector may comprise any nucleic acid molecule capable of transporting another nucleic acid to which it has been linked. A vector may comprise a plasmid, which may be a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors may include cosmids, bacterial artificial chromosomes (BAC) and yeast artificial chromosomes (YAC). A vector may comprise a viral vector, wherein additional DNA segments may be ligated into the viral genome. Some vectors may be capable of autonomous replication in a host cell into which they are introduced (e.g., vectors having an origin of replication which functions in the host cell). Other vectors can be integrated into the genome of a host cell upon introduction into the host cell and are thereby replicated along with the host genome. Some vectors may be capable of directing the expression of genes to which they are operatively linked. Expression may be stable or transient. Stable or transient expression may be achieved through stable or transient transfection, lipofection, electroporation or infection with recombinant viral vectors. Transfection may be the introduction of a heterologous nucleic acid into eukaryote cells, both higher and lower eukaryote cells, as well as yeast and fungal cells. Transfection deliberately introduces nucleic acids into eukaryotic cells artificially to enable the expression or production of proteins using the cell's own machinery or to down-regulate the production of a specific protein by stopping translation.

Introduction of nucleic acids by viral infection may have higher transfection efficiencies than other methods such as lipofection and electroporation. Transfection with viral or non-viral constructs may comprise using adenovirus, lentivirus, Herpes simplex I virus, or adeno-associated virus (AAV) and lipid-based systems. A lipid may be one or more molecules (e.g., biomolecules) that include a fatty acyl group (e.g., saturated or unsaturated acyl chains). A lipid may include oils, phospholipids, free fatty acids, phospholipids, monoglycerides, diglycerides, and triglycerides. Useful lipids for lipid-mediated transfer of the gene may comprise, DOTMA, DOPE, and DC-Choi. Nucleotides may be delivered by neutral or anionic liposomes, cationic liposomes, lipid nanoparticles, ionizable lipids, or any combinations or variations thereof. A preferred construct may comprise viral vectors such as adenoviruses, AAV, lentiviruses, or retroviruses. A viral construct such as a retroviral construct may include at least one transcriptional promoter/enhancer or locus defining element(s), or other elements that control gene expression by other approaches, such as alternate splicing, nuclear RNA export, or post-translational modification of messenger. A vector construct may further comprise a packaging signal, long terminal repeats (LTRs) or portions thereof, or positive and negative strand primer binding sites appropriate to the virus used. A construct may also include a signal sequence for secretion of the peptide from a host cell in which it is placed. A signal sequence may comprise a mammalian signal. Other non-viral vectors can be used such as cationic lipids, polylysine, or dendrimers. An expression construct may comprise the necessary elements for the transcription and translation of an inserted coding sequence. An expression construct may further comprise sequences engineered to enhance stability, production, purification, or yield of the expressed peptide. For example, the expression of a fusion protein or a cleavable fusion protein comprising the MYOD1 and/or myogenin protein of some and a heterologous protein can be engineered. Prokaryotic or eukaryotic cells can be used as host-expression systems to express polypeptides of interest such as microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus (CaMV); tobacco mosaic virus (TMV)) or transformed with recombinant plasmid expression vectors, such as Ti plasmid, containing the coding sequence. Mammalian expression systems can also be used to express polypeptides of interest.

As described herein, forced, transient, non-integrative gene expression can be achieved using various nucleic acid molecules such as messenger ribonucleic acid (mRNA), complementary deoxyribonucleic acid (cDNA), micro RNA (miRNA), transfer RNA (tRNA) mRNA, silencing RNA (siRNA) or any variants, combinations, or analogs thereof. A nucleic acid may be natural in origin or may be a synthetic nucleic acid molecule. Gene expression may be transient, non-integrative such that nucleic acid molecules delivered into a cell are not integrated into the genome of the cell. mRNA introduced into a cell may make a protein by translation which may be sufficient to differentiate a naïve stem cell into a mature cell type. mRNA can be used to differentiate a cell such as with an induced pluripotent stem cell (iPSC) to a skeletal muscle myocyte or transdifferentiate a mature cell such as a fibroblast to a skeletal muscle myocyte. mRNA differentiation protocols may be short (e.g., less than or equal to about 15 days, 14 days, 13 days, 12 days, 10 days, 9 days, 8 days, 7 days, 6 days, 5 days, or less) and may not cause or harbor adverse effects since mRNAs are otherwise degraded and do not integrate with the host cell genome. mRNA may be a single stranded RNA molecule that corresponds to the genetic sequence of a gene and may be read by the ribosome in the process of transcription. mRNA may be complementary to one of the DNA strands of a gene. An mRNA molecule may carry a portion of the DNA code to other parts of the cell for processing. mRNA may be created during transcription wherein a single strand of DNA is decoded by RNA polymerase, synthesizing mRNA.

A nucleic acid molecule may suppress, enhance, or inhibit gene expression in a sequence-specific manner. A nucleic acid molecule may comprise enhancer RNA (eRNA), which may increase expression of a particular gene or set of genes. A nucleic acid molecule may comprise small interfering (siRNA), configured to bind to a gene or gene transcript, thereby inhibiting its expression. siRNA may be a class of short, double stranded RNA non-coding RNA molecules which may interfere with the expression of specific genes with complementary nucleotide sequences. siRNA may interfere with gene expression by degrading mRNA after transcription, preventing translation. In some cases, an siRNA molecule comprises 20-24 base pairs. In some cases, an siRNA molecule comprises a phosphorylated 5′ end and a hydroxylated 3′ end. siRNAs may target complementary mRNA for degradation, thus preventing translation. A nucleic acid molecule may comprise an siRNA precursor, such as a microRNA (miRNA) molecule comprising an siRNA sequence and configured for cleavage upon contact to a cell.

Micro RNA (miRNA) can be small non-coding RNA molecules that function in RNA silencing and post-transcriptional regulation of gene expression. miRNAs base-pair with complementary sequences within mRNA molecules, silencing the mRNA molecules. Silencing may be achieved upon binding of the miRNA to the 3′UTR of the target mRNA through cleavage of the mRNA strand into two pieces, destabilization of mRNA through shortening the poly-A tail, or through inefficient translation of the mRNA into proteins by ribosomes. Modulation of myogenic gene expression may occur through miRNAs. miRNAs that may modulate myogenic gene expression may comprise miR-1, miR-24, miR-26a, miR-27b, miR-29b/c, miR-125b, miR-133, miR-181, miR-206, miR-208b/499, miR-214, miR-221/222, miR-322/424, mi486, or miR-503. These miRNAs may be specifically expressed in cardiac and skeletal muscles under the control of the myogenic transcription factors SRF, MyoD or MEF2 where they may regulate processes of skeletal myogenesis such as myoblast/satellite cell proliferation and differentiation.

Transfer RNA (tRNAs) are adaptor molecules important to translation composed of RNA which serve as a physical link between an mRNA and an amino acid sequence of proteins by carrying an amino acid to the ribosome as directed by a 3-nucleotide codon in a mRNA. tRNAs may be essential for the initiation of protein synthesis by catalyzing ligation of each amino acid to its cognate tRNAs. The translational functions of these entities may be necessary for myogenesis and myogenic differentiation/proliferation. tRNAs that may modulate myogenic gene expression may comprise leucyl-tRNA synthetase, the tRNA gene for lysine, or the tRNA gene for phenylalanine.

cDNA may be a DNA copy synthesized from a single-stranded RNA molecule such as mRNA or miRNA, and produced by reverse transcriptase, a DNA polymer that can use either DNA or RNA as a template. A cDNA can be delivered (e.g., transfected) into a cell to transfer the cDNA that codes for a protein of interest to the recipient cell. A nucleic acid molecule may be delivered to a cell or stem cell to modulate expression of one or more genes in the cells. The modulation may be in a transient and non-integrative manner such that the nucleic acid molecules are not integrated into a genome of the cells. Progenitor cells may be generated following delivery of the cDNA molecules.

Forced human MYOD1 expression may sometimes differentiate human iPSCs and fibroblasts to skeletal muscle myocytes in 7 days with certain constructs. However, protocols used for human cells may not be directly transferrable to non-human species. Additionally, novel mRNA transcripts may need to be produced to improve and guarantee species-specific expression using distinct gene sequences for individual species based on various mRNA expression structures such as in cis-acting elements from 5′ to 3′, cap structure, 5′UTR, coding regions with modified nucleotides, 3′ UTR and a poly-A tails. Species accuracy may improve overall efficiency of the expression system. For example, a bovine viral vector and mRNA sequence in bovine cell culture may provide a more efficient expression system than a human viral vector and mRNA sequence in a bovine cell culture.

In some aspects, the present disclosure provides a method for differentiating stem cells to produce an edible meat product, the method comprising delivering nucleic acid molecules comprising one or more ribonucleic acid (RNA) molecules into the stems cells; modulating expression of one or more genes in the stem cells with aid of the nucleic acid molecules to cause at least a subset of the stem cells to yield one or more progenitor cells following delivery of the nucleic acid molecules, wherein upon modulating, the nucleic acid molecules are not integrated into a genome of the stem cells; culturing the one or more progenitor cells to generate one or more cultured cells; and differentiating the one or more cultured cells to generate one or more terminally differentiated cells to produce the edible meat product. In some cases, the nucleic acid molecules comprise one or more different ribonucleic acid (RNA) molecules. In some cases, the nucleic acid molecules are generated via in vitro transcription. In some cases, the method further comprises delivering a second set of nucleic acid molecules comprising one or more ribonucleic acid (RNA) molecules into cells (e.g., stems cells, mature cells, progenitor cells, or terminally differentiated cells). In some cases, the second set of nucleic acid molecules delivered into the progenitor cells or the cultured cells are different than the nucleic acid molecules delivered into the stem cells. For example, the nucleic acid molecules delivered into the stem cells may encode a myocyte differentiation factor, and the second set of nucleic acid molecules may comprise an siRNA targeting a pluripotency gene to enhance the stability of the progenitor cells. Differentiating the one or more cultured cells to generate one or more terminally differentiated cells to produce the edible meat product may comprise producing a tissue from the one or more terminally differentiated cells.

Cell culturing and differentiating may be performed in a same bioreactor chamber. A bioreactor may be any manufactured device or system which supports a biologically active environment. A bioreactor may be a container suitable for the cultivation of eukaryotic cells, such as mammalian animal cells, or tissues in the context of cell culture. A bioreactor may culture various cell types together, in parallel, or may culture only one cell type singularly. A bioreactor may comprise one vessel or a plurality of vessels and may recycle media used during culture. Culturing at least a subset of progenitor cells or all progenitor cells to generate cultured cells and differentiating at least a subset of the cultured cells to generate terminally differentiated cells to produce an edible meat product may be performed in the same bioreactor chamber or differentiating at least a subset of the cultured cells to generate terminally differentiated cells to produce an edible meat product may be performed in an additional bioreactor.

Under certain conditions, mRNA targeting MYOD alone can be inefficient for differentiating stem cells or trans-differentiating mature cells (e.g., in the production of heterogeneous cell and tissue types). Modulating expression of one or more genes in the stem cells may comprise using one or more RNAs (e.g., two or more different messenger RNAs (mRNAs)) to generate progenitor cells. For example, forced expression of both PAX7 and MYOD1 together may result in a higher percentage of overall skeletal muscle cells in culture than forced expression of just PAX7 or forced expression of just MYOD1. Modulating expression of one or more genes in the stem cells may comprise using one or more messenger RNAs encoding one or more of MYOG, MYF5, MYF6, PAX3, or PAX7 to generate progenitor cells.

Furthermore, suppression of pluripotent genes with silencing RNAs (siRNA) can enhance skeletal muscle formation from iPSCs. The transient modulation of expression of one or more genes in a stem cell may comprise RNA modifications using siRNA(s), or microRNA(s) configured to spontaneously form the siRNA(s) upon cellular uptake. An siRNA may target POUF51 (OCT3/4), KLF4, SOX2 or any variants, combinations, or analogues thereof. siRNA may increase differentiation efficiency, and may enhance differentiated cell stability or viability. For example, an siRNA targeting OCT3/4 (POU5F1), a pluripotent master regulator, may increase the efficiency of MYOD1 mRNA forced expression.

A nucleic acid molecule may comprise an unlocked nucleic acid molecule. An RNA molecule may be modified. A modification to a nucleic acid, such as an RNA molecule, may comprise modification with unlocked nucleic acid monomers (uRNAs). An individual or a plurality of nucleic acids may be modified with a uRNA. A uRNA may be a small RNA molecule found within the splicing speckles and Cajal bodies of the cell nucleus in eukaryotic cells. uRNAs are generally short, around 150 nucleotides in length, and function in processing pre-messenger RNA in the nucleus. uRNAs are abundant and non-coding. uRNAs may remove introns from pre-mRNAs through successive phosphoryl transfer reactions and make up a spliceosome complex, generating a diversity of mRNA isoforms from each coding gene. A uRNA is a ribonucleoside homologue that lacks a C2′-C4′ bond found in ribonucleosides and is therefore flexible. A uRNA may not lock the ribose moiety in the C3′-endo conformation and incorporation of uRNAs into duplexes may be destabilizing. uRNA monomers may be useful in tuning the specificity and potency of siRNAs without affecting cell viability.

The nucleic acid molecules may comprise unlocked nucleic acid molecules. At least one of the nucleic acid molecules may be modified with unlocked nucleic acid monomers. A uRNA may be incorporated at various points along at least one of the nucleic acid molecules, such as at least one of the RNA molecules.

RNA may be chemically modified for example to improve its stability. Eukaryotic mRNA may comprise a coding region flanked by a 5′ and 3′ untranslated regions (UTRs), as well as a 5′ 7-methylguanosine triphosphate cap and a 3′ poly-A tail which may be necessary in mRNA stability and translation. A chemical modification to improve RNA stability may comprise anti-reverse cap analogues, 3′-globin UTR, or poly-A tail modifications. A capped or anti-reverse capped mRNA may have enhanced translational efficiency. Cap analogues may comprise modifications to the 5′ end of an mRNA by addition of 7-Methylguanosine (N⁷-methyl guanosine (m⁷G). Cap analogues may be incorporated in reverse orientation with the methylated G proximal to the RNA which may result in an inability to translate mRNA transcripts. An anti-reverse capped analogue may not be incorporated in reverse orientation as they contain only one 3′-OH group rather than the two 3′-OH groups in the initial cap analogues and may increase translational efficiency over a conventional cap analogue. An anti-reverse capped analogue may comprise a 3′-O-methyl, 3′-H, or 2′-O-methyl modification in the 7-methylguanosine, or N2 modifications (benzyl or 4-methoxybenzyl). Eukaryotic mRNA transcripts include 5′ and 3′ untranslated regions (UTRs) which may comprise regulatory elements. RNA stability and translational efficiency may be improved by incorporating 5′ and 3′ UTRs. A UTR may comprise alpha-globin or beta-globin mRNAs. Beta-globin 5′ and 3′ UTRs may improve translational efficiency and alpha-globin 3′ UTRs may stabilize mRNA. A poly-A tail may be added to the 3′ end of eukaryotic mRNA transcripts during transcription which may regulate mRNA stability and translation synergistically with the m⁷G cap by binding poly-A binding protein forming a complex with the m⁷G cap. A poly-A tail may be encoded on the DNA template from which the mRNA is transcribed, or recombinant poly-A polymerase may be used to extend the mRNA after transcription. Increasing the length of the poly-A tail may increase the efficiency of polysome formation as well as the level of protein expression.

In some aspects, the present disclosure provides a method for generating an edible meat product using stem cells. The method may comprise delivering into the stem cells two or more different types of nucleic acid molecules. Non-limiting examples of nucleic acid molecules which may be delivered into the cells comprise, e.g., messenger ribonucleic acid (mRNA), microRNA (miRNA), transfer RNA (tRNA), silencing RNA (siRNA), enhancer ribonucleic acid (eRNA), complementary deoxyribonucleic acid (cDNA), or any combination or variant thereof. The nucleic acid molecules can be delivered into the cells. The nucleic acid may degrade in the cell. The nucleic acid molecules may not pose any significant adverse effects to the cells. Following delivery of the nucleic acid molecules, expression of one or more genes in the cells may be altered or modulated (e.g., with the aid of or due to the presence of the nucleic acid molecules). The alteration or modulation may comprise enhancing, reducing, or inhibiting the gene expression. The alteration or modulation may be in a transient or non-integrative manner such that the nucleic acid molecules are not integrated into a genome of the stem cells. Such alteration or modulation of gene expression may cause at least a subset of the cells to yield one or more progenitor cells. Some or all of the progenitor cells may subsequently be cultured to generate cultured cells, which cultured cells may be differentiated to generate terminally differentiated cells. The terminally differentiated cells can be used to produce an edible meat product.

The two or more different types of nucleic acid molecules may be generated by an in vitro process. The two or more different types of nucleic acid molecules may comprise mRNA and siRNA. An mRNA may comprise MYOD1, MYOG, MYF5, MYF6, PAX3, PAX7, or any combination or variant thereof. An siRNA may target POUF51 (OCT3/4), KLF4, SOX2, or any combination or variant thereof. The two or more different types of nucleic acid molecules may comprise cDNA and siRNA. A cDNA may comprise MYOD1, MYOG, MYF5, MYF6, PAX3, PAX7, or any combination or variant thereof. The two or more different nucleic acids may comprise a mRNA, cDNA, miRNA, tRNA, siRNA, uRNA, eRNA, or any variant, combinations, or analogs thereof.

One or more genes may be targeted and modulated with one, two, or a plurality of nucleic acid molecules. One or more genes may comprise greater or equal to about 1, 2, 3, 4, 5, 6, 7, 8, 9, 10 genes, or more. Modulating expression of one or more genes in said stem cells may comprise enhancing expression of a first gene of the at least two genes, and inhibiting expression of a second gene of the at least two genes.

RNA transfection may lower the dosing requirements for cell differentiation. Owing to poor cellular uptake and weak effect size, some differentiation factors require frequent dosing and high concentrations to affect cell differentiation. In addition to high costs, intensive dosing regimens can create cytotoxic conditions which lower cell viability. The low dosing requirements of many of the RNA-based differentiation methods disclosed herein can mitigate these cost and toxicity issues, and can confer enhanced stability to differentiated cell populations, further diminishing requirements for ongoing dosing. For example, myoblasts differentiated with a myogenic factor (e.g., MYF5) may require repeated dosing during expansion to maintain their differentiated state, while myoblasts differentiated with a single dose of MYF5-encoding mRNA may be stable throughout expansion. RNA transfection may also facilitate rapid differentiation and cell development. For example, delivery of a single dose of MYOD1-encoding mRNA method may generate muscle tissue from iPSCs after only a short time period (e.g., less than or equal to about 14, 13, 12, 11, 10, 9, 8, 7, 6, 5 days or less).

A cell differentiation method consistent with the present disclosure may comprise delivering nucleic acid molecules comprising one or more ribonucleic acid (RNA) molecules into cells; modulating expression of one or more genes in the cells with aid of the nucleic acid molecules following delivery of the nucleic acid molecules, wherein upon modulating, the nucleic acid molecules are not integrated into a genome of the cells; culturing the cells; and differentiating the cells to generate one or more terminally differentiated cells to produce the edible meat product, wherein the delivering comprises a single instance of contacting the cells with the nucleic acid molecules. A cell differentiation method consistent with the present disclosure may comprise delivering nucleic acid molecules comprising one or more ribonucleic acid (RNA) molecules into cells; modulating expression of one or more genes in the cells with aid of the nucleic acid molecules following delivery of the nucleic acid molecules, wherein upon modulating, the nucleic acid molecules are not integrated into a genome of the cells; culturing the cells; and differentiating the cells to generate one or more terminally differentiated cells to produce the edible meat product, wherein the delivering comprises a plurality of instances of contacting the cells with the nucleic acid molecules. In some cases, the delivering comprises at most two instances of contacting the cells with the nucleic acid molecules. In some cases, the delivering comprises at most three instances of contacting the cells with the nucleic acid molecules. In some cases, the delivering comprises at most four instances of contacting the cells with the nucleic acid molecules. In some cases, the delivering comprises at least one instance of contacting the cells with the nucleic acid molecules. In some cases, the delivering comprises at least two instances of contacting the cells with the nucleic acid molecules. In some cases, the delivering comprises at least three instances of contacting the cells with the nucleic acid molecules. In some cases, the delivering comprises at least four instances of contacting the cells with the nucleic acid molecules. In some cases, two or more instances of contacting the cells with the nucleic acid molecules comprises contacting the cells with different nucleic acid molecules. For example, a first instance of contacting the cells may comprise MYOD1-encoding mRNA and siRNA targeting POUF51, and a second instance of contacting the cells (e.g., 7 days after the first instance of contacting the cells) may comprise MYOD1-encoding mRNA and MYF6-encoding mRNA. In some cases, two or more instances of contacting the cells with the nucleic acid molecules comprises contacting the cells with different quantities of nucleic acid molecules. A second instance of contacting the cells with the nucleic acid molecules may comprise at most 80%, at most 60%, at most 50%, at most 40%, at most 30%, at most 25%, at most 20%, at most 15%, or at most 10% of the amount (e.g., by molar quantity) of nucleic molecules as a first instance of contacting the cells with the nucleic acid molecules. A first instance of contacting the cells with the nucleic acid molecules may comprise at least 120%, at least 150%, at least 200%, at least 250%, at least 300%, at least 400%, or at least 500% of the amount of nucleic acid molecules of all subsequent instances of contacting. For example, iPSCs contacted with 200 nM PAX7 and MYOD1 mRNA may generate myoblasts which require less than 40 nM PAX7 and MYOD1 mRNA to continue efficiently differentiating.

In some cases, the delivering comprises contacting the cells at most once every 3 days. In some cases, the delivering comprises contacting the cells at most once every 5 days. In some cases, the delivering comprises contacting the cells at most once every 7 days. In some cases, the delivering comprises contacting the cells at most once every 10 days. In some cases, the delivering comprises contacting the cells at most once every 14 days.

In some cases, the delivering comprises contacting the cells with at most 20 μM RNA. In some cases, the delivering comprises contacting the cells with at most 10 μM RNA. In some cases, the delivering comprises contacting the cells with at most 5 μM RNA. In some cases, the delivering comprises contacting the cells with at most 2 μM RNA. In some cases, the delivering comprises contacting the cells with at most 1 μM RNA. In some cases, the delivering comprises contacting the cells with at most 500 nM RNA. In some cases, the delivering comprises contacting the cells with at most 200 nM RNA. In some cases, the delivering comprises contacting the cells with at most 100 nM RNA. In some cases, the delivering comprises contacting the cells with at most 50 nM RNA. In some cases, the delivering comprises contacting the cells with at most 20 nM RNA. In some cases, the delivering comprises contacting the cells with at most 10 nM RNA. In some cases, the delivering comprises contacting the cells with at most 5 nM RNA. In some cases, the delivering comprises contacting the cells with at most 2 nM RNA. In some cases, the delivering comprises contacting the cells with at most 1 nM RNA. In some cases, the delivering comprises contacting the cells with 10 nM to 500 nM RNA. In some cases, the delivering comprises contacting the cells with 10 nM to 200 nM RNA. In some cases, the delivering comprises contacting the cells with 20 nM to 200 nM RNA. In some cases, the delivering comprises contacting the cells with 50 nM to 200 nM RNA. In some cases, the delivering comprises contacting the cells with 10 nM to 100 nM RNA. In some cases, the delivering comprises contacting the cells with 20 nM to 100 nM RNA. In some cases, the delivering comprises contacting the cells with 10 nM to 50 nM RNA. In some cases, the delivering comprises contacting the cells with 10 nM to 500 nM of each of a plurality of RNA molecules. For example, the delivering may comprise contacting the cells with 250 nM mRNA encoding MYOD1, 250 nM mRNA encoding PAX7, and 10 nM of siRNA targeting POUF51. In some cases, the delivering comprises contacting the cells with 10 nM to 200 nM of each of a plurality of RNA molecules. In some cases, the delivering comprises contacting the cells with 20 nM to 200 nM of each of a plurality of RNA molecules. In some cases, the delivering comprises contacting the cells with 50 nM to 200 nM of each of a plurality of RNA molecules. In some cases, the delivering comprises contacting the cells with 10 nM to 100 nM of each of a plurality of RNA molecules. In some cases, the delivering comprises contacting the cells with 20 nM to 100 nM of each of a plurality of RNA molecules. In some cases, the delivering comprises contacting the cells with 10 nM to 50 nM of each of a plurality of RNA molecules.

In some cases, the delivering comprises contacting the cells with at most 5 μM mRNA. In some cases, the delivering comprises contacting the cells with at most 2 μM mRNA. In some cases, the delivering comprises contacting the cells with at most 1 μM mRNA. In some cases, the delivering comprises contacting the cells with at most 500 nM mRNA. In some cases, the delivering comprises contacting the cells with at most 200 nM mRNA. In some cases, the delivering comprises contacting the cells with at most 100 nM mRNA. In some cases, the delivering comprises contacting the cells with at most 50 nM mRNA. In some cases, the delivering comprises contacting the cells with at most 20 nM mRNA. In some cases, the delivering comprises contacting the cells with at most 10 nM mRNA. In some cases, the delivering comprises contacting the cells with at most 5 nM mRNA. In some cases, the delivering comprises contacting the cells with at most 2 nM mRNA. In some cases, the delivering comprises contacting the cells with at most 1 nM mRNA.

In some cases, the delivering comprises contacting the cells with at most 500 nM siRNA or miRNA. In some cases, the delivering comprises contacting the cells with at most 200 nM siRNA or miRNA. In some cases, the delivering comprises contacting the cells with at most 100 nM siRNA or miRNA. In some cases, the delivering comprises contacting the cells with at most 50 nM siRNA or miRNA. In some cases, the delivering comprises contacting the cells with at most 20 nM siRNA or miRNA. In some cases, the delivering comprises contacting the cells with at most 10 nM siRNA or miRNA. In some cases, the delivering comprises contacting the cells with at most 5 nM siRNA or miRNA. In some cases, the delivering comprises contacting the cells with at most 2 nM siRNA or miRNA. In some cases, the delivering comprises contacting the cells with at most 1 nM siRNA or miRNA.

Further aspects of the present disclosure provide edible meat products generated from methods disclosed herein. The methods of the present disclosure not only provide humane, resource efficient, and low cost methods for generating edible meat products, but may also be used to generate products with qualities matching or surpassing those of natural meat. Immediately upon the death of an animal, its muscle cells typically begin to undergo apoptosis, autophagy, and necrosis, as well as broader omic changes that can adversely affect the flavor and profile of meat. An edible meat product generated with a method of the present disclosure may comprise controlled omic and morphological profiles more desirable for consumption. An edible meat product generated with a method of the present disclosure may comprise a high degree of cell uniformity (e.g., muscle size, sarcomere and filament development) and alignment. An edible meat product generated with a method of the present disclosure may comprise a multiple cell types in a controlled ratio and/or pattern, such as alternating stripes or layers of multiple cell types. For example, an edible meat product generated with a method of the present disclosure may comprise myocytes and adipocytes in a controlled ratio of 99:1, 98:2, 97:3, 96:4, 95:5, 94:6, 93:7, 92:8, 91:9, 90:10, 89:11, 88:12, 87:13, 86:14, 85:15, 84:16, 83:17, 82:18, 81:19, 80:20, 79:21, 78:22, 77:23, 76:24, 75:25, 70:30, 65:35, 60:40, 55:45, 50:50, 45:55, or 40:60, or any range therein.

An edible meat product generated with a method of the present disclosure may comprise a scaffold or a portion of a scaffold used for differentiation, culturing, or expansion. An edible meat product may comprise at least 1% edible scaffold by weight, at least 2%% edible scaffold by weight, at least 3%% edible scaffold by weight, at least 4% edible scaffold by weight, at least 5% edible scaffold by weight, at least 6% edible scaffold by weight, at least 7% edible scaffold by weight, at least 8% edible scaffold by weight, at least 9% edible scaffold by weight, at least 10% edible scaffold by weight, at least 12% edible scaffold by weight, at least 15% edible scaffold by weight, at least 20% edible scaffold by weight, at least 25% edible scaffold by weight, at least 30% edible scaffold by weight, at least 35% edible scaffold by weight, at least 40% edible scaffold by weight, or at least 50% edible scaffold by weight. An edible meat product may comprise at most 50% edible scaffold by weight, at most 40% edible scaffold by weight, at most 35% edible scaffold by weight, at most 30% edible scaffold by weight, at most 25% edible scaffold by weight, at most 20% edible scaffold by weight, at most 15% edible scaffold by weight, at most 12% edible scaffold by weight, at most 10% edible scaffold by weight, at most 8% edible scaffold by weight, at most 6% edible scaffold by weight, at most 5% edible scaffold by weight, at most 4% edible scaffold by weight, at most 3% edible scaffold by weight, at most 2% edible scaffold by weight, at most 1% edible scaffold by weight, at most. The amount and type of edible scaffold in an edible meat product may affect its flavor, texture, thickness, and strength.

In addition, the intercellular spacing affected by the scaffold may affect the ratio of muscle cell mass to extracellular muscle matrix (ECM). ECM typically accounts for 2-10% of the mass of muscle tissue, and can contribute to undesirable flavor and texture. Muscle cells grown on or within a scaffold may comprise diminished ECM mass relative to in vivo developed muscle cells (for example due to scaffold adhesion), and thereby develop into softer, more flavorful meat. An edible meat product generated with a method of the present disclosure may comprise less than 10% ECM by mass, less than 8% ECM by mass, less than 6% ECM by mass, less than 5% ECM by mass, less than 4% ECM by mass, less than 3% ECM by mass, less than 2% ECM by mass, less than 1% ECM by mass, or less than 0.5% ECM by mass. An edible meat product generated with a method of the present disclosure may comprise a greater mass of edible scaffold than of ECM.

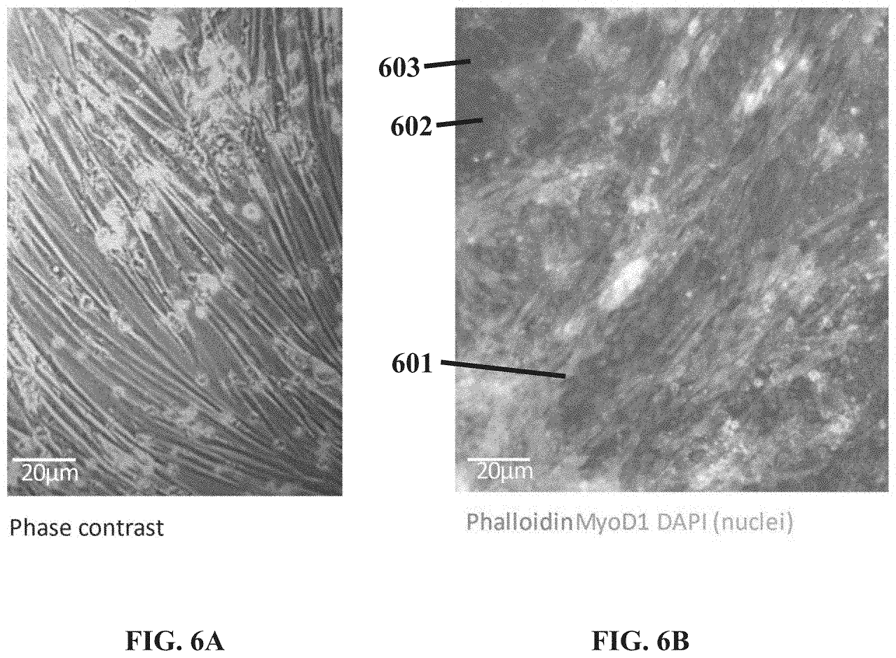

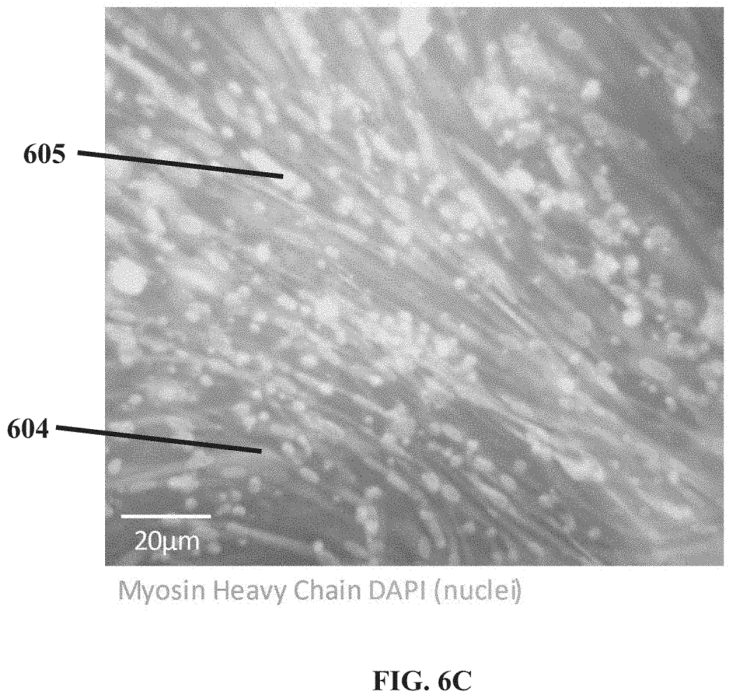

FIG. 6A-C provide three examples of the formation of multinucleated muscle fibers with methods of the present disclosure. The cells are elongated, and express MYOD1 and myosin heavy chain 14 days after differentiation with a single dose of porcine-specific MYOD1 mRNA, showing that the methods of the present disclosure can quickly generate mature muscle tissue. FIG. 6A provides a phase contrast image of the muscle fibers, showing high degrees of multinucleation, elongation, and alignment of the muscle fibers. FIG. 6B provides an image of a cell stain showing actin (phalloidin stain, 601), MYOD (602), and nuclei (DAPI stain, 603). FIG. 6C provides an image of a cell stain showing myosin heavy chain (604) and nuclei (DAPI, 605).

### Biomaterial

Some cultured meat technologies focus on satellite cell cultures with cells grown in two-dimensional flasks or microcarriers in suspension. As provided herein, three-dimensional (3D) scaffolding and tissue engineering platforms may be used to facilitate large-scale growth. A food-safe scaffold may provide structural support and guide the growth of the cultured cells into the desired structure and/or texture analogous with the equivalent food product produced using conventional methods. Culturing a cell or tissue may comprise growing a population of cells on scaffolds within a bioreactor.

In some aspects, the present disclosure provides a method of generating an edible meat product from stem cells. The method may comprise bringing stem cells in contact with a scaffold; subjecting at least a subset of the stem cells to a differentiation process in the presence of the degradable scaffold and with the use of a growth factor or a nucleic acid molecule to generate a tissue; and generating an edible meat product using the tissue, which edible meat product may optionally comprise at least a portion of the scaffold. In some cases, the stem cells are brought into contact with the scaffold prior to being subjected to the differentiation process. In some cases, the stem cells are brought into contact with the scaffold and subjected to the differentiation process at similar times (e.g., within 3 hours of each other). In some cases, the stem cells are subjected to the differentiation process before contacting the scaffold. In some cases, the method further comprises culturing the stem cells to generate cultured stem cells. In some cases, the culturing is subsequent to contacting the scaffold. In some cases, the cultured stem cells are subjected to one or more expansion processes. The scaffold may be engineered to enhance stem cell proliferation, direct cell differentiation into a relevant lineage, or modulate flavor, texture, and tensile elasticity of the final meat product. The scaffold may be degradable. The scaffold may be edible.

Subjecting at least a subset of the stem cells to a differentiation process may comprise use of a plurality of growth factors, a plurality of nucleic acid molecules, or a combination thereof. The plurality of nucleic acid molecules may comprise mRNA encoding a differentiation factor. The plurality of nucleic acid molecules may comprise an interfering RNA (e.g., microRNA or small interfering RNA). The plurality of nucleic acid molecules may comprise transfer RNA. The plurality of nucleic acid molecules may comprise enhancer RNA. Subjecting at least a subset of the stem cells to a differentiation process may comprise use of at least two nucleic acid molecules. The at least two nucleic acid molecules may encode at least two differentiation factors. The at least two nucleic acid molecules may encode at least one differentiation factor and comprise at least one interfering RNA.

A scaffold may enable cell adhesion in a cell culture. A scaffold may enable adherent cells to be grown in a bioreactor system. A bioreactor system may be adherent or a suspension bioreactor system. Culturing stem cells in contact with a degradable scaffold may be performed in a bioreactor chamber and subjecting at least a subset of the cultured stem cells to one or more expansion processes may be performed in a same bioreactor chamber. Culturing stem cells in contact with a degradable scaffold may be performed in a bioreactor chamber and subjecting at least a subset of the cultured stem cells to one or more expansion processes may be performed in an additional bioreactor chamber. One or more of cell culturing, expansion, and differentiation processes may be performed in a same bioreactor chamber, or each may be conducted in a different bioreactor chamber. In some cases, cell culturing is performed in a bioreactor camber and cell expansion is performed in a different bioreactor chamber.

Cultured cells may receive some degree of structural integrity from a scaffold on which the cells may be attached during culturing. Alternatively, a scaffold may not be necessary in suspended cell cultures. Non-adherent cells may not require a substrate or surface for attachment. Cells may have been modified or engineered to no longer require an adherence substrate. For example, hepatocytes are normally adherent cells, but may be modified to no longer require an extracellular matrix for attachment for survival and proliferation. Cultured cells may be grown into cultured tissues that are attached to a support structure such as a two-dimensional or three-dimensional scaffold or support structure. Cultured cells may be grown on a two-dimensional support structure such as a petri-dish where they may form several layers of cells that may be peeled and processed for consumption. Two-dimensional support structures may include porous membranes that allow for diffusion of nutrients from culture media on one side of the membrane to the other side where the cells are attached. In such a composition, additional layers of cells may be achieved by exposing the cells to culture media from both sides of the membrane, for example, cells may receive nutrients through diffusion from one side of the membrane and also from the culture media covering the cells growing on the membrane.

Cultured cells may be grown on, around, or inside a three-dimensional support structure. The support structure may be sculpted into different sizes, shapes, and forms to provide the shape and form for the cultured cells to grow and resemble different types of tissues such as steak, tenderloin, shank, chicken breast, drumstick, lamb chops, fish fillet, lobster tail, etc. The support structure may be a natural or synthetic biomaterial. A biomaterial may comprise any substance intended to interface with biological systems to evaluate, treat, augment, or replace any tissue, organ, or function in a biocompatible manner, such as with a level of acceptable biological response. A biomaterial may interact passively with cells and tissues or may comprise a bioactive material which induces a specific and intended biological response. A biomaterial may comprise a substrate that has been engineered to take a form which alone or as part of a complex system, is used to direct, by control of interactions with components of living systems. A biomaterial may be natural, synthetic, or some combination thereof. A scaffold may be composed of one material or one or more different materials. The support structure may be non-toxic and edible so that they may not be harmful if ingested and may provide additional nutrition, texture, flavor, or form to the final food product. A scaffold may comprise a hydrogel, a biomaterial such as an extracellular matrix molecule (ECM), or biocompatible synthetic material. ECM molecules may comprise proteoglycans, non-proteoglycan polysaccharides, or proteins. A micro-scaffold may be smaller than a conventional tissue culture scaffold which may provide a macroscopic structure and/or shape for the cell population. A micro-scaffold may provide a surface for adherent cells to attach to even while the micro-scaffold itself is in suspension. A micro-scaffold may provide a seed or core structure for adherent cells to attach while remaining small enough to remain in suspension with stirring. The use of micro-scaffolds enables the culturing of adherent cells in a suspension culture which may enable the large-scale production of adherent cells. An edible meat product may be generated using the tissue produced and a degradable scaffold. As an example, the scaffold may be used to guide (as a framework) or facilitate the production the meat product.

A degradable scaffold may comprise a polymeric material. A polymeric material may comprise a natural polymeric material or a synthetic polymeric material. Natural biomaterials may comprise collagen, gelatin, fibrin, alginate, agar, cassava, maize, chitosan, gellan gum, corn-starch, chitin, cellulose, chia (Salvia hispanica) recombinant silk, decellularized tissue (plant or animal), hyaluronic acid, fibronectin, laminin, hemicellulose, glucomannan, textured vegetable protein, heparan sulfate, chondroitin sulfate, tempeh, keratan sulfate, or any combination thereof. A plant-based scaffold may be used for 3D culturing. A plant-based scaffold may comprise scaffolds obtained from plants such as apples, seaweed, or jackfruit. A plant-based scaffold may comprise at least one plant-based material such as cellulose, hemicellulose, pectin, lignin, alginate, or any combination thereof. A textured vegetable protein (TVP), such as textured soy protein (TSP) may comprise a high percentage of soy protein, soy flour, or soy concentrate. TVP and TSP can be used to provide a meat-like texture and consistency to a meat product. Synthetic biomaterials may comprise hydroxyapatite, polyethylene terephthalate, acrylates, polyethylene glycol, polyglycolic acid, polycaprolactone, polylactic acid, their copolymers, or any combination thereof.

A support structure (e.g., a scaffold) may include adhesion peptides, cell adhesion molecules, or other growth factors covalently or non-covalently associated with the support structure. Cell recognition sites may promote cell adhesion and migration. Cell recognition sites may comprise sequences such as Arg-Gly-Asp (RGD) or Arg-Glu-Asp-Val sequences. A synthetic polymeric material may comprise a polyethylene glycol biomaterial comprising an arginylglycylaspartic (RGD) motif. A meat product comprising scaffolding material may be seasoned to taste like meat (e.g., using various salts, herbs, and/or spices). A scaffold may be comprised of a cell or tissue culture product. For example, cartilage derived from chondrocytes may form an underlying support layer or structure together with a support structure. Afterwards, muscle cells or fat cells, or both, may be seeded onto the chondrocyte layer. The interaction of muscle cells and chondrocytes may provide regulatory signals required for tissue formation.

A support structure may be formed as a solid or semisolid support. A support structure may comprise a solid non-porous structure or a porous structure, for example, high porosity may provide maximal surface area for cell attachment. Porous scaffolds may allow cell migration or infiltration into the pores. A porous scaffold may be edible. A porous scaffold may comprise a natural biomaterial or a synthetic biomaterial, textured protein. A porous scaffold may have an average pore diameter. An average pore diameter of the porous scaffold may range from 20 micrometers (μm) to 1000 μm, 20 μm to 900 μm, 20 μm to 800 μm, 20 μm to 700 μm, 20 μm to 600 μm, 20 μm to 500 μm, 20 μm to 400 μm, 20 μm to 300 μm, 20 μm to 200 μm, 20 μm to 100 μm, 50 μm to 1000 μm, 50 μm to 900 μm, 50 μm to 800 μm, 50 μm to 700 μm, 50 μm to 600 μm, 50 μm to 500 μm, 50 μm to 400 μm, 50 μm to 300 μm, 50 μm to 200 μm, 50 μm to 100 μm, 100 μm to 1000 μm, 100 μm to 900 μm, 100 μm to 800 μm, 100 μm to 700 μm, 100 μm to 600 μm, 100 μm to 500 μm, 100 μm to 400 μm, 100 μm to 300 μm, 100 μm to 200 μm, 500 μm to 1000 μm, 500 μm to 900 μm, 500 μm to 800 μm, 500 μm to 700 μm, or 500 μm to 600 μm. An average pore diameter of the porous scaffold may range from about 20 μm to about 1000 μm. An average pore diameter may be less than 20 μm or may be larger than 1000 μm.

A scaffold may degrade during cell culturing or differentiation, increasing the space available for cells to aggregate or cluster within the scaffold. Additionally or alternatively, a scaffold may be configured to degrade in response to cell growth or aggregation. During cell culturing or differentiation, a scaffold may degrade at an average rate of at least 0.25% per day, at least 0.5% per day, at least 1% per day, at least 2% per day, at least 3% per day, at least 4% per day, at least 5% per day, at least 6% per day, at least 8% per day, at least 10% per day, at least 12% per day, at least 15% per day, or at least 20% per day (e.g., measured as a loss of mass). During cell culturing or differentiation, an average pore diameter of a scaffold may increase by at least 0.25% per day, at least 0.5% per day, at least 1% per day, at least 2% per day, at least 3% per day, at least 4% per day, at least 5% per day, at least 6% per day, at least 8% per day, at least 10% per day, at least 12% per day, at least 15% per day, or at least 20% per day. For example, the glycosidic bonds of an alginate scaffold comprising cells may degrade at a rate of about 0.5% per day due to the mechanical stress imposed by the cells, the conditions of the media, or a combination thereof.

A soft, porous material may be preferable with an adequate microstructure and stiffness for the cell type of interest. A scaffold may confer mechanical properties to improve the texture and mouthfeel of a meat product. A scaffold may also confer mechanical properties to encourage proliferation, migration, growth, or differentiation of a desired cell type from a precursor cell. A mechanical property may comprise compression, expansion, strain, stretch, elasticity, shear strength, shear modulus, viscoelasticity, or tensile strength. A scaffold may comprise a material with suitable mechanical properties and degradation kinetics for the desired tissue type that is generated from the cells. For example, a softer surface may be needed in the differentiation and culture of adipocytes as compared to myocytes.

A scaffold may be produced by transforming a material. A scaffold fabrication method may comprise a physical and/or chemical performed on a material to render them usable for cell or tissue culture. Not all biomaterials may be suitable for a given fabrication method or a biomaterial may need to be modified to enable their use in a fabrication method. A scaffold fabrication method may comprise electrospinning, phase separation, freeze drying, lithography, printing, extrusion, self-assembly, solvent casting, textile technologies, material injections, laser sintering, phase separation, porogen leaching, gas foaming, fiber meshing, supercritical fluid processing, or additive manufacturing.

A support structure may comprise a degradable scaffold. A degradable scaffold may be configured to facilitate cell expansion in a culture vessel, such as a bioreactor chamber. A degradable scaffold may be configured to facilitate cell expansion inside a bioreactor chamber. Stem cells may be cultured in the presence of a degradable scaffold to create cultured stem cells. Stem cells may be cultured into cultured stem cells and cultured stem cells may be subjected to one or more expansion processes to generate expanded stem cells in the presence of a degradable scaffold. A degradable scaffold may degrade at approximately an equal rate to tissue formation. A degradable material may enable remodeling and/or elimination of the scaffold in the cultured food product. For example, in some cases, a 3D scaffold that shapes cultured myocytes into the shape of a steak may biodegrade after the myocytes expand to fill up the interior space of the scaffold. The scaffold may also comprise a material that remains in the cultured food product. For example, a portion of a collagen scaffold providing support to cultured myocytes may remain in the final steak to provide texture and continuing structural support in the cultured food product. A scaffold may comprise materials that do not biodegrade and/or remain in the cultured food product for consumption. For example, certain materials can be used to generate the scaffolds in order to confer a particular structure, texture, taste, or other desired property without degradation. A scaffold may comprise a material with texture-modifying properties.

Scaffolds of various compositions can be used to produce a desired texture and/or consistency in the final food product. A natural biomaterial such as a gellan gum, corn starch, chia, alginate, gelatin, chondroitin, fibrinogen, or cassava material may produce a desired texture, consistency, or flavor profile to a final food product. A scaffold may comprise a filler or binder material for providing texture to the food product or may be a filler or binder material for providing texture to a final food product. A scaffold material may biodegrade such that the finished food product no longer has any scaffold structures remaining. For example, a population of cells may be seeded onto a scaffold in a bioreactor. As the cells adhere to the scaffolds and proliferate, the scaffolds gradually biodegrade until all that remains are the clumps of cells that are now adhered to each other and the extracellular matrix materials that they have secreted. A scaffold can be used to guide the structure of the resulting cultured food product and may remain in the food product for consumption by a human. For example, a scaffold for the proliferation of myocytes may comprise a gellan gum material. This material may be engineered such that it only partially biodegrades by the time the meat product is produced in culture. The gellan gum may remain in the meat product acting as a filler and as a texture and flavor enhancer.

### Bioreactor

Cells may be cultured and expanded to a desired quantity such as in a scalable manner using bioreactors to enable large-scale production. A bioreactor apparatus may provide a scalable method for differentiating and expanding stem cells into tissue and with the requisite growth needed for industrial production. Further, the mechanical conditioning of such an apparatus may provide a uniform method of producing a bio-artificial muscle with that simulates standard meat in terms of its appearance, texture, and flavor at a competitive price. For example, some methods of producing cultured meat for human consumption comprise: a) obtaining a population of self-renewing cells derived from an animal; b) culturing the population of self-renewing cells in culture media comprising scaffolds within a bioreactor; c) inducing differentiation in the population of cells to form at least one of terminally differentiated cells such as myocytes and adipocytes within a bioreactor; and d) culturing the cells into tissue within a bioreactor thus processing the population of cells into meat for human consumption.

A bioreactor system may comprise at least one bioreactor, bioreactor tank, or reactor chamber. For example, a bioreactor system may comprise at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 reactor chambers. A bioreactor system may comprise about 1 reactor chamber to more than 1,000 reactor chambers. A bioreactor system may comprise about 1 reactor chamber or more than 1 reactor chambers. A reactor chamber may have an internal volume suitable for large-scale cell culture. A reactor chamber may have an internal volume of about 0.1 Liters (L) to about 1,000,000 L. A reactor chamber may have an internal volume of less than 1 L or an internal volume of greater than 1,000,000 L. A reactor chamber may have an internal volume of about less than 1 L to about 1 L, about 1 L to about 10 L, about 1 L to about 50 L, about 1 L to about 100 L, about 1 L to about 500 L, about 1 L to about 1,000 L, about 1 L to about 5,000 L, about 1 L to about 10,000 L, about 1 L to about 50,000 L, about 1 L to about 1,000,000 L, about 10 L to about 50 L, about 10 L to about 100 L, about 10 L to about 500 L, about 10 L to about 1,000 L, about 10 L to about 5,000 L, about 10 L to about 10,000 L, about 10 L to about 50,000 L, about 10 L to about 1,000,000 L, about 50 L to about 100 L, about 50 L to about 500 L, about 50 L to about 1,000 L, about 50 L to about 5,000 L, about 50 L to about 10,000 L, about 50 L to about 50,000 L, about 50 L to about 1,000,000 L, about 100 L to about 500 L, about 100 L to about 1,000 L, about 100 L to about 5,000 L, about 100 L to about 10,000 L, about 100 L to about 50,000 L, about 100 L to about 1,000,000 L, about 500 L to about 1,000 L, about 500 L to about 5,000 L, about 500 L to about 10,000 L, about 500 L to about 50,000 L, about 500 L to about 1,000,000 L, about 1,000 L to about 5,000 L, about 1,000 L to about 10,000 L, about 1,000 L to about 50,000 L, about 1,000 L to about 1,000,000 L, about 5,000 L to about 10,000 L, about 5,000 L to about 50,000 L, about 5,000 L to about 1,000,000 L, about 10,000 L to about 50,000 L, about 10,000 L to about 1,000,000 L, or about 50,000 L to about 100,000 L or more than 1,000,000 L.

As described above or elsewhere herein, cell culturing, differentiation and/or expansion may each be conducted in a separate bioreactor chamber. In some examples, all processes (e.g., culturing, expansion, differentiation) may be performed in the same bioreactor chamber. As another example, cell culturing may be performed in a bioreactor chamber and expansion and/or differentiation may be performed in an additional bioreactor chamber. The bioreactor chamber or the additional bioreactor chamber may comprise a plurality of bioreactor chambers. Each of the plurality of the bioreactor chambers or the additional bioreactor chambers may be configured to facilitate a specific process (e.g., culturing, expansion, differentiation). In some cases, a subset or all of the cultured stem cells from the bioreactor chamber may be directed to a plurality of additional bioreactor chambers to perform a plurality of expansion processes, which may comprise greater than or equal to about 1, 2, 3, 4, 5, 6, 7, 8, 9, 10, 15, 20, 25, 30, 35, 40, 45, 50 expansion processes, or more. The plurality of expansion processes may be performed sequentially, simultaneously, or a combination thereof.

In some aspects, the present disclosure provides a method for differentiating stem cells to produce an edible meat product. The method may comprise culturing one or more progenitor cells to generate one or more cultured cells and differentiating the one or more cultured cells to generate one or more terminally differentiated cells which can be used for producing an edible meat product. As described above or elsewhere herein, the culturing one or more progenitor cells to generate one or more cultured cells and differentiating the one or more cultured cells to generate one or more terminally differentiated cells may be performed in a same bioreactor chamber or may be performed in different bioreactor chambers.

A bioreactor system may be suitable for large-scale production of cultured cells for generation of food products. Cells may be cultured on a batch basis. Alternatively, or in combination, cells may be cultured continuously. In both batch and continuous cultures, fresh nutrients may be supplied to ensure the appropriate nutrient concentrations for producing the desired food product. As an example, in a fed-batch culture, nutrients (e.g. fresh culture media) is supplied to the bioreactor, and the cultured cells remain in the bioreactor until they are ready for processing into the finished food product. In a semi-batch culture, a base media may be supplied to the bioreactor and may support an initial cell culture, while an additional feed media is then supplied to replenish depleted nutrients. A bioreactor system may produce at least a certain quantity of cells per batch. A bioreactor system may produce a batch of about 1 billion cells to about 100,000,000 billion cells. A bioreactor system may produce a batch of at least about 1 billion cells. A bioreactor system may produce a batch of about 100,000,000 billion cells. A bioreactor system may produce a batch of less than 1 billion cells to about 1 billion cells, about 1 billion cells to 10 billion cells, about 1 billion cells to about 50 billion cells, about 1 billion cells to about 100 billion cells, about 1 billion cells to about 500 billion cells, about 1 billion cells to about 1,000 billion cells, about 1 billion cells to about 5,000 billion cells, about 1 billion cells to about 10,000 billion cells, about 1 billion cells to about 100,000 billion cells, about 1 billion cells to about 1,000,000 billion cells, about 1 billion cells to about 10,000,000 billion cells, about 1 billion cells to about 100,000,000 billion cells, about 10 billion cells to about 50 billion cells, about 10 billion cells to about 100 billion cells, about 10 billion cells to about 500 billion cells, about 10 billion cells to about 1,000 billion cells, about 10 billion cells to about 5,000 billion cells, about 10 billion cells to about 10,000 billion cells, about 10 billion cells to about 100,000 billion cells, about 10 billion cells to about 1,000,000 billion cells, about 10 billion cells to about 10,000,000 billion cells, about 10 billion cells to about 100,000,000 billion cells, about 50 billion cells to about 100 billion cells, about 50 billion cells to about 500 billion cells, about 50 billion cells to about 1,000 billion cells, about 50 billion cells to about 5,000 billion cells, about 50 billion cells to about 10,000 billion cells, about 50 billion cells to about 100,000 billion cells, about 50 billion cells to about 1,000,000 billion cells, about 50 billion cells to about 10,000,000 billion cells, about 50 billion cells to about 100,000,000 billion cells, about 100 billion cells to about 500 billion cells, about 100 billion cells to about 1,000 billion cells, about 100 billion cells to about 5,000 billion cells, about 100 billion cells to about 10,000 billion cells, about 100 billion cells to about 100,000 billion cells, about 100 billion cells to about 1,000,000 billion cells, about 100 billion cells to about 10,000,000 billion cells, about 100 billion cells to about 100,000,000 billion cells, about 500 billion cells to about 1,000 billion cells, about 500 billion cells to about 5,000 billion cells, about 500 billion cells to about 10,000 billion cells, about 500 billion cells to about 100,000 billion cells, about 500 billion cells to about 1,000,000 billion cells, about 500 billion cells to about 10,000,000 billion cells, about 500 billion cells to about 100,000,000 billion cells, about 1,000 billion cells to about 5,000 billion cells, about 1,000 billion cells to about 10,000 billion cells, about 1,000 billion cells to about 100,000 billion cells, about 1,000 billion cells to about 1,000,000 billion cells, about 1,000 billion cells to about 10,000,000 billion cells, about 1,000 billion cells to about 100,000,000 billion cells, about 5,000 billion cells to about 10,000 billion cells, about 5,000 billion cells to about 100,000 billion cells, about 5,000 billion cells to about 1,000,000 billion cells, about 5,000 billion cells to about 10,000,000 billion cells, about 5,000 billion cells to about 100,000,000 billion cells, about 10,000 billion cells to about 100,000 billion cells, about 10,000 billion cells to about 1,000,000 billion cells, about 10,000 billion cells to about 10,000,000 billion cells, about 10,000 billion cells to about 100,000,000 billion cells, about 100,000 billion cells to about 1,000,000 billion cells, about 100,000 billion cells to about 10,000,000 billion cells, about 100,000 billion cells to about 100,000,000 billion cells, about 1,000,000 billion cells to about 10,000,000 billion cells, about 1,000,000 billion cells to about 100,000,000 billion cells, or about 10,000,000 billion cells to about 100,000,000 billion cells or more than 100,000,000 billion cells.

A bioreactor system may produce a batch of cultured cells during a certain time period. For example, in some cases, a bioreactor system may produce a batch of cultured cells at least once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or more. A bioreactor system may produce a batch of cultured cells having at least a certain mass. Sometimes, the mass is measured as dry weight with excess media or supernatant removed. A bioreactor system may produce a batch of cultured cells of about 1 kilogram (kg) to about 100,000 kg. In certain instances, a bioreactor system produces a batch of at least about 1 kg. A bioreactor system may produce a batch of about 100,000 kg or more than 100,000 kg. A bioreactor system may produce a batch of about less than 1 kg to 1 kg, about 1 kg to about 5 kg, about 1 kg to about 10 kg, about 1 kg to about 20 kg, about 1 kg to about 30 kg, about 1 kg to about 40 kg, about 1 kg to about 50 kg, about 1 kg to about 100 kg, about 1 kg to about 500 kg, about 1 kg to about 1,000 kg, about 1 kg to about 5,000 kg, about 1 kg to about 100,000 kg, about 5 kg to about 10 kg, about 5 kg to about 20 kg, about 5 kg to about 30 kg, about 5 kg to about 40 kg, about 5 kg to about 50 kg, about 5 kg to about 100 kg, about 5 kg to about 500 kg, about 5 kg to about 1,000 kg, about 5 kg to about 5,000 kg, about 5 kg to about 100,000 kg, about 10 kg to about 20 kg, about 10 kg to about 30 kg, about 10 kg to about 40 kg, about 10 kg to about 50 kg, about 10 kg to about 100 kg, about 10 kg to about 500 kg, about 10 kg to about 1,000 kg, about 10 kg to about 5,000 kg, about 10 kg to about 100,000 kg, about 20 kg to about 30 kg, about 20 kg to about 40 kg, about 20 kg to about 50 kg, about 20 kg to about 100 kg, about 20 kg to about 500 kg, about 20 kg to about 1,000 kg, about 20 kg to about 5,000 kg, about 20 kg to about 100,000 kg, about 30 kg to about 40 kg, about 30 kg to about 50 kg, about 30 kg to about 100 kg, about 30 kg to about 500 kg, about 30 kg to about 1,000 kg, about 30 kg to about 5,000 kg, about 30 kg to about 100,000 kg, about 40 kg to about 50 kg, about 40 kg to about 100 kg, about 40 kg to about 500 kg, about 40 kg to about 1,000 kg, about 40 kg to about 5,000 kg, about 40 kg to about 100,000 kg, about 50 kg to about 100 kg, about 50 kg to about 500 kg, about 50 kg to about 1,000 kg, about 50 kg to about 5,000 kg, about 50 kg to about 100,000 kg, about 100 kg to about 500 kg, about 100 kg to about 1,000 kg, about 100 kg to about 5,000 kg, about 100 kg to about 100,000 kg, about 500 kg to about 1,000 kg, about 500 kg to about 5,000 kg, about 500 kg to about 100,000 kg, about 1,000 kg to about 5,000 kg, about 1,000 kg to about 100,000 kg, or about 5,000 kg to about 100,000 kg or more than 100,000 kg.

Cell and tissue culture may occur in one or a plurality of bioreactors or bioreactor chambers throughout growth, expansion, and differentiation. There may be 1, 2, 3, 4, 5, 6, 7, 8, 9, 10, or more than 10 bioreactors or bioreactor chambers used in cell or tissue culture. A bioreactor system comprises about 1 reactor chamber to about 5 reactor chambers, about 1 reactor chamber to about 10 reactor chambers, about 1 reactor chamber to about 20 reactor chambers, about 1 reactor chamber to about 50 reactor chambers, about 1 reactor chamber to about 100 reactor chambers, about 1 reactor chamber to about 200 reactor chambers, about 1 reactor chamber to about 300 reactor chambers, about 1 reactor chamber to about 400 reactor chambers, about 1 reactor chamber to about 500 reactor chambers, about 1 reactor chamber to about 1,000 reactor chambers, about 5 reactor chambers to about 10 reactor chambers, about 5 reactor chambers to about 20 reactor chambers, about 5 reactor chambers to about 50 reactor chambers, about 5 reactor chambers to about 100 reactor chambers, about 5 reactor chambers to about 200 reactor chambers, about 5 reactor chambers to about 300 reactor chambers, about 5 reactor chambers to about 400 reactor chambers, about 5 reactor chambers to about 500 reactor chambers, about 5 reactor chambers to about 1,000 reactor chambers, about 10 reactor chambers to about 20 reactor chambers, about 10 reactor chambers to about 50 reactor chambers, about 10 reactor chambers to about 100 reactor chambers, about 10 reactor chambers to about 200 reactor chambers, about 10 reactor chambers to about 300 reactor chambers, about 10 reactor chambers to about 400 reactor chambers, about 10 reactor chambers to about 500 reactor chambers, about 10 reactor chambers to about 1,000 reactor chambers, about 20 reactor chambers to about 50 reactor chambers, about 20 reactor chambers to about 100 reactor chambers, about 20 reactor chambers to about 200 reactor chambers, about 20 reactor chambers to about 300 reactor chambers, about 20 reactor chambers to about 400 reactor chambers, about 20 reactor chambers to about 500 reactor chambers, about 20 reactor chambers to about 1,000 reactor chambers, about 50 reactor chambers to about 100 reactor chambers, about 50 reactor chambers to about 200 reactor chambers, about 50 reactor chambers to about 300 reactor chambers, about 50 reactor chambers to about 400 reactor chambers, about 50 reactor chambers to about 500 reactor chambers, about 50 reactor chambers to about 1,000 reactor chambers, about 100 reactor chambers to about 200 reactor chambers, about 100 reactor chambers to about 300 reactor chambers, about 100 reactor chambers to about 400 reactor chambers, about 100 reactor chambers to about 500 reactor chambers, about 100 reactor chambers to about 1,000 reactor chambers, about 200 reactor chambers to about 300 reactor chambers, about 200 reactor chambers to about 400 reactor chambers, about 200 reactor chambers to about 500 reactor chambers, about 200 reactor chambers to about 1,000 reactor chambers, about 300 reactor chambers to about 400 reactor chambers, about 300 reactor chambers to about 500 reactor chambers, about 300 reactor chambers to about 1,000 reactor chambers, about 400 reactor chambers to about 500 reactor chambers, about 400 reactor chambers to about 1,000 reactor chambers, or about 500 reactor chambers to about 1,000 reactor chambers or more than 1,000 reactor chambers.

Growth, culturing, expansion, and differentiation may be concurrent or in parallel in the same or in different bioreactors or bioreactor chambers. For example, a bioreactor system may be designed such that there are two bioreactors in which iPSC expansion occurs and four bioreactors in which iPSC differentiation occurs. Cells may be grown within a first bioreactor of scalable size for a period of approximately 7 days. Cells may be grown for approximately, 1, 2, 3, 4, 5, 6, 7, 8, 9, 10, 15, 20, 25, 30, 35, 40, 45, 50, 60, 70, 80, 90, or more than 90 days. One or more expansion processes may comprise passaging at least a subset or all cultured stem cells. Cells may be passaged to a subsequent bioreactor approximately four times the size of the first bioreactor of scalable size. A subsequent bioreactor may be 2, 3, 4, 5, 6, 7, 8, 9, 10, or more than 10 times the size of the first bioreactor of scalable size. A subsequent bioreactor may be less than 10, 9, 8, 7, 6, 5, 4, 3, 2, or 1 time the size of the first bioreactor of scalable size. Cultured cells may be “split” or “passaged” approximately every 7 days, but the cells can be split more often or less often, depending on the specific needs and circumstances of the culture. For example, the cells may be split every 1, 2, 3, 4, 5, 6, 7, 8, 9, 10, 11, 12, 13, 14 or more days, or any time frame in between. The cell split or passaging may comprise the collection of cells from a previous culture and subsequent transfer of the collected (harvested) cells into a new cell culture vessel. Passaging may allow the cells to continue to grow in a healthy cell culture environment. Processes and methods of cell culture passaging may involve the use of enzymatic or non-enzymatic methods to disaggregate cells that have clumped together during their growth expansion. Passaging may comprise passing an enzyme over at a subset or all cultured stem cells to detach them from a surface of the degradable scaffold. Cells can be passaged using enzymatic, non-enzymatic, or manual dissociation methods prior to and/or after contact with the defined medium. Non-limiting examples of enzymatic dissociation methods include the use of proteases such as trypsin, TrypLE, collagenase, dispase, and accutase. When enzymatic passaging methods are used, the resultant culture can comprise a mixture of singlets, doublets, triplets, and clumps of cells that vary in size depending on the enzymatic method used. A non-limiting example of a non-enzymatic dissociation method is a cell dispersal buffer or ethylenediaminetetraacetic acid (EDTA). The choice of passaging method may be influenced by the choice of cell type, extracellular matrix or a biomaterial scaffold, if one is present.

To passage cells from one bioreactor to the next, media may be drained from the bioreactor shelves and may be replaced by phosphate buffered saline (PBS) to wash the cells. PBS may be run over the cells such that each shelf in the bioreactor may be submerged in PBS for at least 15 seconds, after which the PBS may be removed and discarded. Each shelf in the bioreactor may be submerged in PBS for about at least 1 second (s), 2 s, 3 s, 4 s, 5 s, 6 s, 7 s, 8 s, 9 s, 10 s, 15 s, 20 s, 25 s, 30 s, 35 s, 40 s, 45 s, 50 s, 60 s, 70 s, 80 s, 90 s or more than 90 s. Each shelf in the bioreactor may be submerged for less than about 1 s. An enzyme or chemical solution such as EDTA in PBS may be passed over the cells to detach the cells from their surface of adhesion, for example a shelf, scaffold, or surface in the bioreactor. The cells may be incubated in the enzyme or chemical solution for a period of time, such as 4-8 minutes (min), before the solution is removed and discarded. The cells may be incubated in the enzyme or chemical solution for about at least 1 minute (min.)-2 min., 1 min.-3 min., 1 min.-4 min., 1 min.-5 min., 1 min.-6 min., 1 min.-7 min., 1 min.-8 min., 1 min.-9 min., 1 min.-10 min., or 1 min.-more than 10 min., 2 min.-3 min., 2 min.-4 min., 2 min.-5 min., 2 min.-6 min., 2 min.-7 min., 2 min.-8 min., 2 min.-9 min., 2 min.-10 min., 2 min.-more than 10 min., 3 min.-4 min., 3 min.-5 min., 3 min.-6 min., 3 min.-7 min., 3 min.-8 min., 3 min.-9 min., 3 min.-10 min., 3 min.-more than 10 min., 4 min.-5 min., 4 min.-6 min., 4 min.-7 min., 4 min.-8 min., 4 min.-9 min., 4 min.-10 min., 4 min.-more than 10 min., 5 min.-6 min., 5 min.-7 min., 5 min.-8 min., 5 min.-9 min., 5 min.-10 min., 5 min.-more than 10 min., 6 min.-7 min., 6 min.-8 min., 6 min.-9 min., 6 min.-10 min., 6 min.-more than 10 min., 7 min.-8 min., 7 min.-9 min., 7 min.-10 min., 7 min.-more than 10 min., 8 min.-9 min., 8 min.-10 min., 8 min.-more than 10 min., 9 min.-10 min., or 9 min.-more than 10 min. Cells may be incubated in an enzyme or chemical solution for less than 1 min or more than 10 min. Media from a media storage tank may be used to collect the detached cells by passing media over the cells and the cells in the media, may be collected in an additional tank to be passed to a centrifuge/cell filter system to isolate the cell and colony pieces from the media. A condensed cell/media solution may then be further mixed with media from a media storage tank as it flows into a subsequent bioreactor using decreasing flow rates to enable equal coating of bioreactor shelves. Cells may be separated using centrifugation or through an alternative method such as cell filtration which may separate cells of the size of a cell of interest out, such as an iPSC.

Cells may be expanded in a subsequent bioreactor for approximately 7 days or may be expanded in a same bioreactor for approximately 7 days. Cells may be expanded for approximately, 1, 2, 3, 4, 5, 6, 7, 8, 9, 10, 15, 20, 25, 30, 35, 40, 45, 50, 60, 70, 80, 90, or more than 90 days. Cells may be further passaged into one or a plurality of bioreactors which may be 2, 3, 4, 5, 6, 7, 8, 9, 10, or more than 10 times the size of a previous bioreactor of scalable size. A subsequent bioreactor may be less than 10, 9, 8, 7, 6, 5, 4, 3, 2, or 1 time the size of the previous bioreactor of scalable size thus splitting the cells by a ratio dependent on the size of the bioreactors and resultant density of the cultured cells.

Differentiation may occur in the final bioreactor or may occur in a previous bioreactor. Differentiation of a stem cell or progenitor cell into a terminally differentiated cell may take approximately 14-21 days or more. Differentiation of a stem cell or progenitor cell into a terminally differentiated cell may take 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or more than 90 days. Differentiation of a stem cell or progenitor cell into a terminally differentiated cell may take less than 90, 80, 70, 60, 50, 45, 40, 35, 30, 25, 20, 19, 18, 17, 16, 15, 14, 13, 12, 11, 10, 9, 8, 7, 6, 5, 4, 3, 2, 1, or less than one day. For example, a mesenchymal stem cell may be differentiated into a tissue comprised of skeletal muscle myocytes after 17 days of appropriate culture in a bioreactor. When a mature tissue has been produced, it may be removed from the system by pulling out each layer as a draw and extracting the food product. A mature tissue may comprise mature skeletal muscle fibers which may be drawn out by extracting the meat.

Expansion and differentiation phases may use one or different types of media. Media and growth conditions may be optimized using different media, temperatures, conditions, or compositions. One or multiple media storage tanks may be used to store one or multiple types of media. Media storage tanks may comprise an area for storage of differentiation factors or small molecules in solution. Media storage tanks may be temperature controlled and individual tanks in a plurality of tanks may store media at different temperatures. For example, media may be stored at 4° C. and differentiation factors to be mixed with media stored at −20° C. Differentiation factors, media components, or media stored at freezing or below freezing temperatures may be thawed automatically and added into an appropriate media storage tank when required. Some media components may remain fresh for several weeks while some differentiation factors or nucleotides may be maintained as frozen as they may degrade rapidly in less than 24 hours. Media may comprise a serum or may utilize a serum free media. Culture medium may comprise maintenance media, differentiation media, steatotic media, proliferation media, or any other media formulation. Culture medium may be refreshed about every 1, 2, 3, 4, 5, 6, 7, 8, 9, 10, 11, 12, 13, 14, 15, 16, 17, 18, 19, 20, 21, 22, 23, 24, or more than 24 hours, or any fraction thereof. In additional examples, the medium may be refreshed less often such as, but not limited to, every 1.1, 1.2, 1.3, 1.4, 1.5, 1.6, 1.7, 1.8, 1.9 or every 2 or more days, or any time frame in between.

In some aspects, the present disclosure provides a method for producing an edible meat product. The method may comprise modulating expression of one or more genes in stem cells in a transient and non-integrative manner using one or more or two or more different compositions (e.g., ectopic differentiation factors) to generate progenitor cells, culturing at least a subset of the progenitor cells to generate cultured cells, and differentiating at least a subset of the cultured cells to generate terminally differentiated cells to produce the edible meat product. The culturing and differentiating may be performed in the same bioreactor chamber or may be performed different bioreactor chambers. A terminally differentiated cell may comprise muscle cells, fat cells, bone cells, endothelial cells, smooth muscle cells, neural cells somite cells, or a combination thereof.

Ectopic differentiation factors may induce differentiation in a transient and non-integrative manner using non-native induction through biochemical systems. Ectopic differentiation factors may comprise nucleic acids, polypeptides, small molecules, growth factors, or any combination thereof. A cultured stem cell or progenitor cell may be differentiated by arresting the cell cycle of the stem cell or progenitor cell. Ectopic differentiation factors may arrest the cell cycle of cells by reducing or removing growth factors. Ectopic differentiation factors may arrest the cell cycle of cells through reducing or removing growth factors from a subset of cultured cells. Growth factors may be reduced or removed from a subset of cultured cells. Self-renewal and pluripotency of stem cells may be governed by extrinsic signals mediated by an endogenous pluripotency gene regulatory network consisting of a set of core transcription factors such as Oct3/4 or Sox2. Transcription factor interactions may regulate genomic functions by establishing both negative and positive feedback loops and transcription by recruiting activators and repressors to modulate the transcriptional machinery. Maintaining stem cell characteristics of self-renewal and differentiation in pluripotent stem cells may require distinct extrinsic signaling pathways including leukemia inhibitory factor (LIF), FGF/extracellular signal-regulated kinase (ERK) pathway, Wnt/glycogen synthase kinase 3 (GSK3), and transforming growth factor-beta (TGF-β) signaling. Growth factors which may influence the differentiation of stem or progenitor cells may comprise LIF, FGF, BMP, activin, MAPK, and TGF-β. Leukemia Inhibitory Factor may be a polyfunctional glycoprotein with actions on a broad range of tissue and cell types, including induction of differentiation in a number of myeloid leukemic cell lines, suppression of differentiation in normal embryonic stem cells, stimulation of proliferation of osteoblasts and haemopoietic cells. LIF may be necessary in establishing iPSCs from differentiated somatic cells. The addition of LIF to cell culture may improve the reprogramming of iPSCs from somatic cells as well as aid in the maintenance of stem cell proliferation. Activated fibroblast growth factor (FGF) signaling may sustain stem cells capabilities by promoting self-renewing proliferation and inhibiting cellular senescence. The removal of LIF may lead to the reversible conversion of embryonic stem cells from a naïve state to four FGF receptors/ERK-committed early differentiation states with features characteristic of primed pluripotency. Bone morphogenetic proteins (BMPs) through the SMAD-inhibitor of differentiation pathway with LIF may retain stem cell self-renewal and differentiation potential in stem cells. Inhibition of MAPK/ERK signaling pathway activation downstream of FGF signaling may improve stem cell stability and stemness. The FGF4/ERK signaling pathway activation may be necessary in multi-lineage differentiation of stem cells. FGF2 and Activin may enhance the expression of Oct4, thereby allowing the reversion from primed to naïve state of pluripotency in stem cells. TGFβ/activin/nodal signals via SMAD2/3 may be associated with stem cell pluripotency and may be required for the maintenance of primed stem cells and progenitor cells. Arresting the cell cycle of stem or progenitor cells may occur by reducing or removing serum levels in a solution in which the culturing is conducted. For example, replacing media comprising serum molecules with serum-free media may arrest the cell cycle of an iPSC and enhance the differentiation potential of the cell.

A bioreactor system may be scalable for large-scale cell culture. A bioreactor system may comprise a reactor chamber for culturing cells. A bioreactor system may comprise an element for agitation of the contents of the reactor chamber or otherwise mechanical or electrical stimulation of the contents of the reactor chamber. Fresh media may be added into the reactor chamber via at least one input port. Depleted media or effluent may be removed from the reactor chamber via at least one output port. Oxygen, carbon dioxide, and/or other gases may be introduced through at least one input gas port. An input gas port may be coupled to an aerator positioned inside the reactor chamber. A bioreactor system may comprise at least one sensor for monitoring the reactor chamber which may be in communication with a control unit (e.g. a computer). A bioreactor system may facilitate production of cultured tissues for human consumption. A bioreactor may comprise a reactor chamber comprising a plurality of scaffolds or surfaces that provide adhesion surfaces for cellular attachment, a population of self-renewing cells cultivated within bioreactor, a first source providing at least one maintenance media comprising components for maintaining the population of self-renewing cells without spontaneous differentiation, and a second source providing at least one differentiation media comprising components for differentiating the population of self-renewing cells into a specific lineage. A reactor chamber may comprise a plurality of scaffolds or shelves which enable adherence of certain adherent cell. A series of scaffolds, shelves, or culture surfaces may be present on which cells may attach and grow. A bioreactor system may comprise at least one degradable, food safe, scaffold. These shelves may be arranged such that the shelves are angled in opposite angles to each other. The angle of the shelves may be less than 1°, about 1°, 2°, 3°, 4°, 5°, 6°, 7°, 8°, 9°, 10°, or more than a 10° angle.

There may be perfusion laminar flow, aided by gravity, of media over the cells. Media may flow from the top of the bioreactor to the bottom of the bioreactor, where media may be recycled. When media reaches the last shelf of the bioreactor, the run off may be pumped upwards against gravity through a diaphragm system enabling the dialysis of waste products from the media. Media after filtering may be replenished of lost nutrients and other media components before re-entering the bioreactor from the top of the reactor to utilize gravity. Removal of gasses, such as carbon dioxide and the replenishment of gasses, such as oxygen, may be performed during recycling. Gases may be managed within the media using a custom system, or a commercial system alongside a dialysis membrane or plurality of dialysis membranes.

Culturing stem cells to generate cultured stem cells and subjecting at least a subset of cultured stem cells to one or more expansion processes to generate expanded stem cells may comprise directing a medium through a bioreactor chamber and an additional bioreactor chamber to facilitate culturing stem cells or the one or more expansion processes. The medium may be under continuous laminar flow or oscillatory flow. The medium may be configured to promote cell culturing or expansion. The medium may be directed out of an additional bioreactor chamber. The medium may be filtered during direction from an additional bioreactor chamber to remove undesired components from the medium, thereby generating a filtered medium. The filtering may remove ammonium, lactate, alanine, methyl glyoxylate, and other cellular waste products. The filtering may minimally impact nucleic acid and differentiation factor concentration. The filtered medium may be recycled back into the bioreactor chamber. Filtering media may comprise using any type of filter that can remove contaminants and impurities such as carbon filtering or zeolite filtering. Media recycling may comprise a closed-loop perfusion system, such as a dialysis unit permitting physiological addition of nutrients and removal of toxins. Temperature within a recycling system may be maintained at a constant temperature, such as 37° C., or may comprise a varied temperature. Media running throughout the reactor may contain the required dissolved oxygen or a gap above the media and below a shelf may be utilized for air circulation. A perfusion system may comprise a primary tissue perfusion circuit and a secondary dialysis circuit for nutrient and toxin exchange. A primary circuit may comprise culture medium perfusate that is recirculated using a pump through a tissue growth chamber, a membrane oxygenator, a heat exchanger, or a bubble trap. A pump may be constant, oscillatory, or peristaltic. A membrane oxygenator may be gassed with a mixture of 80% 0₂/5% CO₂/15% N₂ maintaining constant pH. Some or all of the perfusate may be diverted to a secondary circuit. A secondary circuit may comprise a dialyzer, such as a hollow fiber dialyzer. A secondary circuit may dialyze the perfusate, such as by using a counter-current exposure to protein-free dialysate and recirculate the perfusate through a filter using a pump.

Delivery of a perfusion solution may occur via a fluidic circuit which may be controlled by a controller by the use of a pump in a delivery system. Delivery of a perfusion solution may be constituted to enrich the perfusion solution by a culture medium and one or more gaseous media, such as oxygen, carbon dioxide or nitrogen. The perfusion solution may be operatively coupled to a reservoir that enriches the perfusion solution by the culture medium and by one or more gaseous media, such as with an oxygenator. The gas balance in the media may comprise a mixture of oxygen from about 21% to about 95%, Carbon dioxide from about 0% to about 10% and balanced to 100% by Nitrogen. For example, a bioreactor may provide a mixture of media of about 80% Oxygen about 5% carbon dioxide and about 15% nitrogen held at 37° C. at pH 7.2.

Media may be recycled at a predetermined time interval or based on an established benchmark such as cell density or composition of the conditioned medium. There may be a waste medium vessel or a fresh medium vessel in fluid communion with the bioreactor chambers. A waste medium vessel may collect media that is not recycled to facilitate draining and replacement of media in a controlled manner. A waste medium vessel may be in fluid communion to a dialyzer to filter waste medium and return the treated medium to the system. During media recycling a percentage of the medium may be removed and replaced with fresh basal medium added and/or used media removed, purified, and returned to a bioreactor chamber or fresh medium vessel. The medium to be exchanged may comprise at least 1%, at least 2.5%, at least 5%, at least 7.5%, at least 10%, at least 12.5%, at least 15%, at least 17.5%, at least 20%, or more than 20% of the original volume in the bioreactor chambers. The medium to be exchanged may comprise less than 1% of the original volume in the bioreactor chambers.

Culture conditions in a bioreactor may comprise static, stirred, or dynamic flow conditions. A bioreactor may be scaled in size to produce greater volume of cells or to allow greater control over the flow of nutrients, gases, metabolites, and regulatory molecules. A bioreactor may provide physical and mechanical signals such as compression, stretch, or alterations in flow to stimulate cells to produce specific biomolecules or to differentiate into a specific cell type. Unlike tissues derived from whole animals, tissues grown ex vivo or in vitro may have never been exercised (e.g. never been used to move a leg) and thus may have differences in flavor or texture without stimulation which may mimic the effects of exercise. A cell or tissue culture, or whole meat product may be exposed to a stimulus to increase the similarity in texture or flavor between meat grown ex vivo or meat derived from a whole animal. A cell or tissue culture may be exposed to a mechanical or electrical stimulus. A mechanical stimulus may comprise compression, expansion, shear flow, stretch, oscillatory flow, or dynamic stretch. An electrical stimulus may comprise an electric or oscillating current. Exposing the cultured cells, tissue, or the meat products in vitro to a mechanical or electrical stimulus may increase the growth rate of cultured cells ex vivo. The mechanical or electrical stimulus may be applied to stem or progenitor cells or to cells after they have differentiated from their precursor cells.

Cultured meat may comprise a mixed population of cells, such as myocytes and adipocytes. Progenitor cells such as pre-adipocytes or satellite cells may be isolated from a source and may have some self-renewal capacity. These self-renewing cells may be cultured, expanded, and subsequently differentiated in a bioreactor. In some cases, a heterogeneous composition of self-renewing cells may be cultured together, or they may be cultured separately until after differentiation when they may be co-cultured together at a certain ratio to produce a desired ratio in a final meat product. A population of cells may be induced to differentiate into different cell types in the same culture. For example, some cells from a progenitor cell may form into adipocytes and some form into myocytes. These myocytes and adipocytes may be cultured separately, and subsequently mixed or may be homogeneously mixed in equal proportions. The myocytes and adipocytes may be heterogeneously mixed in unequal proportions. For co-culturing or processing, the myocytes and adipocytes may be combined at a certain ratio or proportion. For example, in some cases, myocytes and adipocytes may be combined at a ratio of at least 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 35:1, 40:1, 45:1, 50:1, 60:1, 70:1, 80:1, 90:1, or at least 100:1, respectively.

A meat product may comprise a meat having a certain ratio of fast twitch and slow twitch muscle cells and/or fibers. A meat product may comprise myocytes or skeletal muscle cells having a certain ratio or proportion of fast twitch (type II) and slow twitch (type I) muscle fibers. Slow twitch muscle fibers may exhibit low-intensity contractions fueled by the oxidative pathway and demonstrate relatively higher endurance, while fast twitch muscle fibers may have higher intensity contractions fueled by the glycolytic pathway. Fast twitch muscles may be characterized by high glycolytic and anaerobic muscle fibers. The ratio of fast twitch and slow twitch muscle fibers in muscle tissue may play a role in the taste, color, texture, and other culinary properties of the meat.

The bioreactor system may enable the culturing of cells for food production in a pathogen-free environment. Cells may be grown in a culture environment free of dangerous contaminants that affect human health. Cell culture plates, flasks, and bioreactors may provide cell culture conditions free of dangerous pathogens (e.g. H1N1), parasites, heavy metals, or toxins (e.g. bacterial endotoxins, pesticides, etc.). A cell culture system may not utilize antibiotics, in contrast to traditional livestock agriculture. A differentiation factor, media component or nucleotide molecule, or otherwise induction modality used in cell culture may be transient or may be removed before the cells or tissues are processed into a food product.

An edible meat product may be in a unit form of approximately or greater than 50 grams (g). An edible meat product may be in a unit form of at least about 1 g, 2 g, 3 g, 4 g, 5 g, 6 g, 7 g, 8 g, 9 g, 10 g, 15 g, 20 g, 25 g, 30 g, 35 g, 40 g, 45 g, 50 g, 60 g, 70 g, 80 g, 90 g, 100 g, 150 g, 200 g, 250 g, 300 g, 350 g, 400 g, 450 g, 500 g, 600 g, 700 g, 800 g, 900 g, 1000 g, or more than 1000 g. An edible meat product may be in a unit form of less than 1 g. A hamburger patty for example, may have a precooked weight of 85 g-113 g (3-4 ounces) if served diner style or 198 g-226 g (7-8 ounces) if served in a heavier pub-style.

### Computer Systems

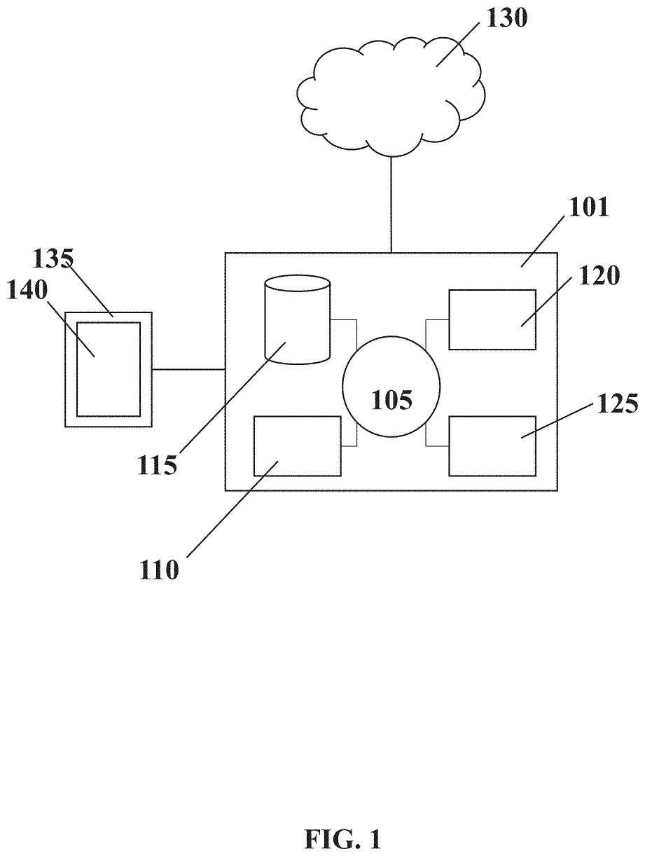

The present disclosure provides computer systems that are programmed to implement methods of the disclosure. FIG. 1 shows a computer system 101 that is programmed or otherwise configured to perform the methods described herein. The computer system 101 can regulate various aspects of the present disclosure, such as, for example, determining a ratio of media supplied to a culture in a bioreactor. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 1130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user (e.g., a cellular network). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine-readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140, for example, determining a ratio of media supplied to a culture or the flow rate of media during recycling in a bioreactor. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, determine a ratio of media supplied to a culture or the flow rate of media during recycling in a bioreactor.

### EXAMPLES

The following examples are included to further describe some aspects of the present disclosure and should not be used to limit the scope of the disclosure.

### Example 1

### Overview of Cell Culture Methodology in Producing an Edible Meat Product

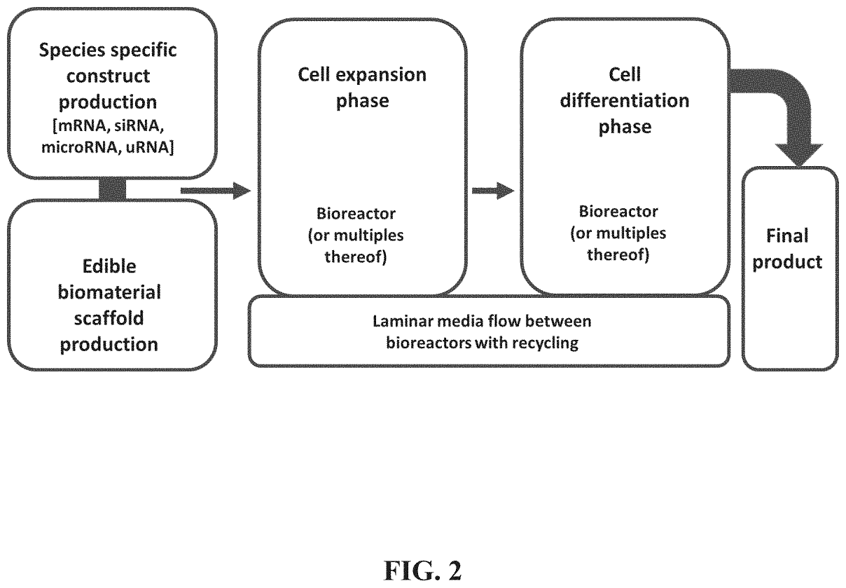

As illustrated in FIG. 2, an edible biomaterial scaffold is produced either separately to or in parallel to developing species-specific construct production for mRNA, siRNA, miRNA, or uRNAs. Cells are seeded on the edible scaffold and the scaffold placed in a bioreactor. Cells are then expanded in a bioreactor or multiple bioreactors. These reactors are either a single vessel bioreactor or may comprise a plurality of bioreactor vessels. An expansion bioreactor is in fluid contact with laminar media flow and media recycling with either a single vessel bioreactor or plurality of bioreactor vessels for cell differentiation. Cell differentiation may comprise an alteration of media, genetic manipulation, or ectopic differentiation factors being added during culture. Differentiated cells are then expanded further until they form tissue on the scaffolds, at which point the tissue may be removed from the reactor by drawing it out where it may directly be used as an edible meat product or may be processed further into a meat product.

### Stem Cell Expansion and Differentiation in Culture for an Edible Meat Product

Porcine iPSCs are maintained and expanded in iPSC medium (KO DMEM supplemented with 10% KO serum, 10 nanograms per milliliter ng/mL bFGF2, 10 ng/mL human LIF, 0.1 mM non-essential amino acids, 2 mM glutamine) on geltrex-coated plates. Cells are seeded onto geltrex coated plates and coverslips, and differentiation commenced at 60% confluence as follows.

24 hours prior, cells are fed with OPTI-MEM reduced serum medium (ThermoFisher), supplemented with 10 uM Y27632 (ROCK inhibitor, Sigma Aldrich). Lipofectamine Stem Transfection Reagent (ThermoFisher) is used in accordance with the manufacturer's instructions. Briefly, 75 milligrams/milliliter (mg/mL) mRNA is mixed with OPTI-MEM reduced serum medium (ThermoFisher) and combined with lipofectamine stem reagent for 10 minutes at room temperature before being added to the cells. Cells are incubated at 37° C. for 24 hours, and the process repeated for 3 consecutive days. On the 4^{th} day, cells are switched to myogenic medium (KO DMEM supplemented with 10% KO serum, 0.1 mM non-essential amino acids, 2 mM glutamine, 0.1 mM β-mercaptoethanol) for maturation and expansion. Cells are taken for analysis between 7- and 21-days post treatment.

Analysis may be conducted using immunohistological staining. Cells on coverslips may be fixed with 4% paraformaldehyde overnight at 4° C. Cells are then incubated at room temperature for 2 hours (or overnight at 4° C.) with blocking agent (PBS+1% Triton-X+10% normal goat serum). Primary antibodies are all added directly the blocking serum at 1:1000, overnight at room temperature. Coverslips are washed 4× in PBS with rocking, and secondary antibodies added at 1:5000 in blocking serum for 2-4 hours at room temperature, protected from light. Primary antibodies used were rabbit myosin heavy chain/MYH3 (ab124205, Abcam); rabbit MYOD1 (ab203383, Abcam); mouse PAX7 (ab199010, Abcam). Secondary antibodies used are goat anti-rabbit IgG H&L (Alexa Fluor 488) (ab150077, Abcam); goat anti-mouse IgG H&L (Alexa Fluor 568) (ab175473, Abcam). Coverslips are thoroughly washed with PBS 4-5 times before being mounted onto glass slides using Antifade Mounting Medium with DAPI (H-1200, Vectashield) in preparation for microscopy. Analysis is conducted using a Leica LAS X Widefield System and Leica Application Suite X (LAS X).

Analysis by PCR to determine gene expression is conducted. Cell lysis is achieved using TE buffer (10 mM Tris-HCL, 1 mM EDTA, pH 8)+1% Sodium Dodecyl Sulphate (SDS). Protein is digested using Proteinase K (200 μg/mL) at 56° C. for 10 min. Precipitate DNA with 0.2M sodium chloride and 100% absolute ethanol. 5 μL of DNA used for each PCR reaction as below using the primer sequences outlined in TABLE 1. PCR was performed with 1 minute and 94° C. denaturation steps, 2 minute and 55° C. annealing steps, and 3 minute and 72° C. extension steps.

TABLE 1
Exemplary primer sequences and

| TARGET | SEQUENCE |
|---|---|
| MYOD1-F1 | (SEQ ID NO: 1) AGCACTACAGTGGCGACTCA |
| MYODI-R1 | (SEQ ID NO: 2) GCTCCACTATGCTGGACAGG |
| MYOD1-F2 | (SEQ ID NO: 3) CCTACTGTGGGCCTGCAAG |
| MYOD1-R2 | (SEQ ID NO: 4) GGATCTCCACCTTGGGCAAC |
| PAX7-F | (SEQ ID NO: 5) CCGTGTTTCCCATGGTTGTG |
| PAX7-R | (SEQ ID NO: 6) GAGCACTCGGCTAATCGAAC |
| GAPDH-F | (SEQ ID NO: 7) ATCACTGCCACCCAGAAGACT |
| GAPDH-R | (SEQ ID NO: 8) CATGCCAGTGAGCTTCCCGTT |
| MYOGENIN-F | (SEQ ID NO: 9) CTACAGGCCTTGCTCAGCTC |
| MYOGENIN-R | (SEQ ID NO: 10) AGTTGTGGGCGTCTGTAGG |

Other mRNAs/miRNAs/siRNAs may be used in permutations to this methodology. Experimental changes may use the same materials and methods, but different compounds may be introduced.
Transient Expression of MYOD1 in Porcine iPSCs

Human MYOD1 is transiently expressed in porcine iPSCs for 3 days using Lipofectamine Stem Transfection reagent. Cells are matured for a further 7 days. Following this maturation, 60% of cells are immunoreactive for either MYOD1 or MyHC (myosin heavy chain). MYOD1 can be expressed in porcine cells, and as a consequence can result in the differentiation of iPSCs to skeletal muscle myocytes. The cells expressed SOX2 show that the early differentiation stage may still be in the window of pluripotency; it may be expected that maturation of the muscle progenitor cells may result in an increase in myogenic markers and a decrease or loss of progenitor stem cell markers. This may be observed in all developmental stages of differentiation and expected. It can be compared to controls using small molecules to differentiate porcine iPSCs to skeletal muscle myocytes, for which there is a working model with 60-70% efficiency rate.

Induction of Cell Differentiation Using mRNA, cDNA, or siRNA

Cells (iPSCs/fibroblasts) are transfected daily with components (e.g. mRNA, siRNA, cDNA, miRNA) between 1-7 days. GFP/RFP/YFP mRNA, or scrambled siRNA are used as a transfection control. Transfection is carried out using either of the following technologies: traditional chemical based methods (e.g. Lipofection), non-chemical methods (e.g. electroporation or nucleofection), nanoparticle methods (e.g. liposome, polymer nanoparticles, micelles, or lipid-nanoparticles), or by magnet assisted transfection.

Cessation of transfection simultaneous with a reduced serum media directs cells down a myogenic lineage, with maturation of cultures over a course of 14-50 days promoting the formation of multinucleated myotubes.

Transfections are carried out in 2D (with or without biomaterial) or 3D (including but not limited to: spheroid, embryoid bodies, suspension or adherent, with or without biomaterial) culture conditions. Maturation of cultures are carried out in the described 2D or 3D conditions, with or without biomaterial, or with or without electrical stimulation or contractile tension forces to promote maturation of myogenic fibers.

The diverse nature of nucleotides affects the delivery method chosen as can be seen in the difference of nucleotide lengths, double vs single stranded nucleic acids, and the dose range of nucleotides: Silencing RNA (siRNA): 20-40 bps, double stranded RNA (dsRNA) molecule, Messenger RNA (mRNA): range of 500 bp-2-4 kbp, single stranded RNA (ssRNA) molecule Dose range of nucleotides: 0.5 μg/mL-50 μg/mL per nucleotide (DNA, RNA, mRNA, siRNA) For example, mRNA and siRNA may be delivered together using a nanoparticle transfection option.

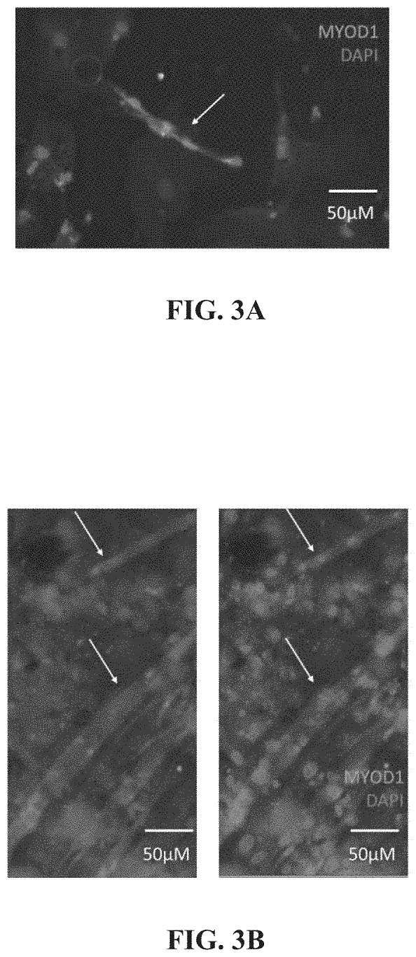

Analysis at set checkpoints is carried out using molecular biology techniques. PCR (polymerase chain reaction) is used to check for transcription factors, such as a decrease in progenitor markers OCT3/4, SOX2, and increased expression of myogenic markers PAX7, MYOD1, Myogenin, MYF5, MYF6, Desmin, myosin heavy chain, and myosin light chain as well as controls. IHC (immunohistochemistry) uses the primary antibodies to detect protein expression of Myosin Heavy Chain, MYOD1, Desmin, PX7, Myogenin, as well as controls. As can be seen in FIG. 3, multinucleated MYOD1 expressing muscle fibers form ten days after differentiation with MYOD mRNA and at 30 days after differentiation with MYOD mRNA, multinucleated, aligned MYOD1 expressing muscle fibers form.

### Cell Culture Using a Scalable Bioreactor

A bioreactor system is designed such that there are two bioreactors in which iPSC expansion occurs and four bioreactors in which iPSC differentiation occurs.

Cells are first grown within the first bioreactor of a size x for a period of approximately 7 days. This approximate time value comes from experience culturing these cells on plastic plates within a lab incubator. At this time, cells are passaged to a bioreactor of size 4× based on approximate splitting values used in the lab. The iPSCs are further expanded within this 4× bioreactor for 7 days. The cells are then further passaged into four 4×bioreactors, to split the cells by a ratio of 1:4 again. It is in these final four bioreactors that differentiation is carried out. The approximate time to differentiate these cells to produce mature skeletal muscle fibers is estimated to be 14-21 days and further. Once the mature skeletal muscle fibers have been produced, they are removed from the system by pulling out each layer as a draw and extracting the meat. This part of the design in particular is subject to change.

As the expansion and differentiation phases call for two different kinds of media, 2 media storage tanks are required. Media within these tanks is stored at 4° C. and differentiation factors to be mixed with the media is stored at −20° C., which are thawed automatically and added into the appropriate media storage tank when required. The reason for this is that media components may remain fresh for at least 2 weeks, whereas some differentiation factors and small molecules may be maintained as frozen as they degrade in less than 24 hours.

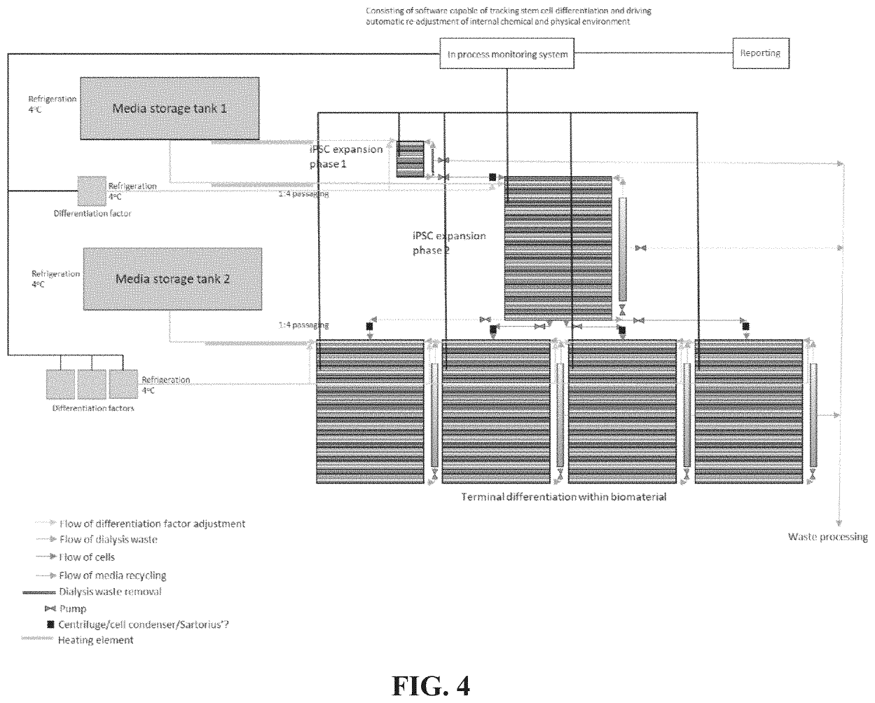

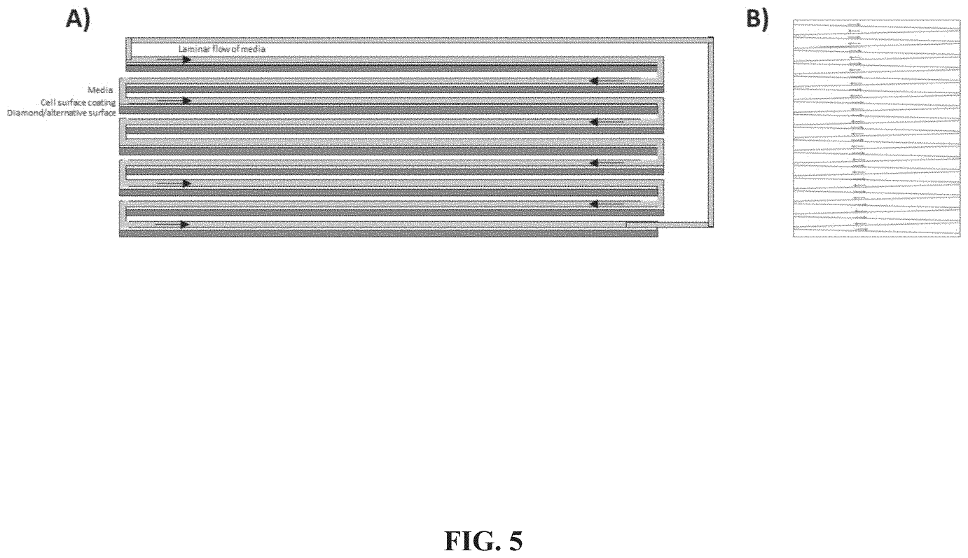

As can be seen in FIG. 4, within the bioreactor, a series of shelves or culture surfaces are present on which the cells attach and grow. These shelves are arranged such that the shelved are angled (estimated 3° to 6° angle) in opposite angles to each other. There is perfusion laminar flow, as can be seen in FIG. 5, aided by gravity, of media over the cells. Once the media, which flows from the top of the bioreactor to the bottom of the bioreactor reaches the bottom, media is recycled. As can be seen in FIG. 5A, the composition of each shelf is made of diamond for its biocompatibility and diagrammed in blue. Media is shown in pink and flow of media with arrows. A thin yellow layer between the media and shelf is shown, which indicated the cell surface coating of vitronectin. The cells are grown on top of the cell surface coating and the media flows over them. As can be seen in FIG. 5 B direction of flow of media represented by arrows throughout each bioreactor and orientation of the shelves represented by horizontal lines.

### Media Recycling, Perfusion, and Re-Introduction of Lost Components

After the initial passaging of cells into each bioreactor, the media within the bioreactor is recycled, rather than replaced daily. The media is under continuous laminar flow, such that when the media reaches the last shelf of the bioreactor, the runoff is pumped upwards against gravity through a diaphragm system (shown in FIG. 4 as the orange rectangle next to each bioreactor enabling dialysis of waste products from the media. After dialysis, the media is then replenished of lost nutrients and other media components before re-entering the bioreactor at the top of the reactor to take advantage of gravity. The media components lost, along with waste products, from the system through dialysis are replaced. CO₂ removal from the system and the replenishment of O₂ levels are an important consideration. Gasses are managed within the media using a membrane contactor system alongside the dialysis membranes.

Passaging the Cells from One Bioreactor to the Next

To passage cells from one bioreactor to the next, the media is drained from the bioreactor shelves and replaced by PBS, after with a short delay of 3-20 seconds occurs to wash the cells at the same time. PBS is run over the cells such that each shelf has been submerged in PBS for 15 seconds, after which the PBS may be removed and discarded through a waste pipe. An enzyme or chemical such as EDTA in PBS (1:1000) may then be passed over the cells to detach the cells from the surface of the plate. The cells may be incubated in the enzyme/chemical solution for 4-8 minutes, before this solution is removed and discarded through the same waste pipe. Media from the media storage tank may then be used to collect the detached cells by passing media over the cells at force (at a higher flow rate) and the cells, in the media, may be collected in an additional tank (pre-separation/centrifugation tank) to be passed to the centrifuge/cell filter system to isolate the cell and colony pieces from the media. The condensed cell/media solution is then further mixed with media from media storage tank 2 as it flows into the next bioreactor using decreasing flow rates to ensure the cell surfaces on each shelf are coated equally. Cells are separated with centrifugation or with an alternative such as cell filtration which separates cells of the size of iPSCs. A bioreactor prototype is 1 Liters (L) in volume while the final manufacturing system is 3750 L in internal volume.

### NUMBERED EMBODIMENTS

Embodiments contemplated herein include embodiments P1 to P112.

Embodiment P1. A method for differentiating or transdifferentiating cells to produce an edible meat product, the method comprising: (a) delivering nucleic acid molecules comprising one or more ribonucleic acid (RNA) molecules into said cells; (b) modulating gene expression of said cells with aid of said nucleic acid molecules or expression products thereof, to differentiate or transdifferentiate at least a subset of said cells to generate one or more target cells following delivery of said nucleic acid molecules, wherein upon said modulating, said nucleic acid molecules are not integrated into a genome of said cells; and producing said edible meat product using at least partially said one or more target cells generated in (b).

Embodiment P2. The method of Embodiment 1, wherein said nucleic acid molecules comprise two or more different RNA molecules.

Embodiment P3. The method of Embodiment 1 or 2, wherein said cells comprise animal cells.

Embodiment P4. The method of Embodiment 3, wherein said animal cells comprise porcine cells.

Embodiment P5. The method of any one of Embodiments 1-4, wherein (c) comprises producing a tissue from said one or more target cells.

Embodiment P6. The method of Embodiment 5, wherein said tissue comprises muscle tissue, fat tissue, neural tissue, vascular tissue, epithelial tissue, connective tissue, bone or a combination thereof.

Embodiment P7. The method of any one of Embodiments 1-6, wherein said one or more target cells comprise at least two different types of cells.

Embodiment P8. The method of Embodiment 7, further comprising co-culturing said at least two types of target cells to generate a three-dimensional tissue.

Embodiment P9. The method of any one of Embodiments 1-8, wherein said one or more target cells comprise muscle cells, fat cells, somite cells, neural cells, endothelial cells, smooth muscle cells, bone cells, or a combination thereof.

Embodiment P10. the method of any one of Embodiments 1-9, wherein said RNA molecules comprise MYOD1, MYOG, MYF5, MYF6, PAX3, or PAX7, or any combination or variant thereof.

Embodiment P11. The method of any one of Embodiments 1-10, wherein said nucleic acid molecules comprise unlocked nucleic acid molecules.

Embodiment P12. The method of any one of Embodiments 1-11, wherein at least one of said RNA molecules is modified with unlocked nucleic acid monomers (uRNAs).

Embodiment P13. The method of Embodiment 12, wherein said uRNAs are incorporated at various points along said at least one of said RNA molecules.

Embodiment P14. The method of any one of Embodiments 1-13, wherein at least one of said RNA molecules is chemically modified to improve its stability.

Embodiment P15. The method of Embodiment 14, wherein chemical modifications to said at least one of said RNA molecules comprise anti-reverse cap analogues, 3′-globin UTR, poly-A tail modifications, or any combination thereof.

Embodiment P16. The method of any one of Embodiments 1-15, wherein said RNA molecules comprise messenger RNA (mRNA), microRNA (miRNA), transfer RNA (tRNA), silencing RNA (siRNA), or a combination thereof.

Embodiment P17. The method of Embodiment 16, wherein said nucleic acid molecules further comprise complementary deoxyribonucleic acid (cDNA) molecules.

Embodiment P18. The method of any one of Embodiments 1-17, wherein said nucleic acid molecules are synthetic nucleic acid molecules.

Embodiment P19. The method of any one of Embodiments 1-18, wherein said nucleic acid molecules are delivered to said cells with neutral or anionic liposomes, cationic liposomes, lipid nanoparticles, ionizable lipids, or any combination or variation thereof.

Embodiment P20. The method of any one of Embodiments 1-19, wherein said nucleic acid molecules are delivered in a single dose to said cells.

Embodiment P21. The method of any one of Embodiments 1-20, wherein said nucleic acid molecules are delivered in at least two doses to said cells.

Embodiment P22. The method of Embodiment 21, wherein individual doses of said at least two doses are delivered at least 3 days apart.

Embodiment P23. The method of Embodiment 21 or 22, wherein individual doses of said at least two doses comprise different nucleic acid molecules.

Embodiment P24. The method of any one of Embodiments 1-23, wherein said nucleic acid molecules are delivered at a concentration of at most 500 nM.

Embodiment P25. The method of any one of Embodiments 1-24, wherein said nucleic acid molecules comprise siRNA targeting POUF51 (OCT3/4), KLF4, SOX2, or any combination or variant thereof.

Embodiment P26. The method of any one of Embodiments 1-25, wherein said cells comprise stem cells, mature cells, or a combination thereof.

Embodiment P27. A method of generating an edible meat product from cells, comprising: (a) bringing said cells in contact with a scaffold; (b) subjecting at least a subset of said cells to a differentiation or a transdifferentiation process in the presence of said scaffold and with the use of a growth factor or a nucleic acid molecule, to thereby generate a tissue; and (c) producing said edible meat product using said tissue.

Embodiment P28. The method of Embodiment 27, wherein said scaffold is degradable, and wherein said edible meat product optionally comprises at least a portion of said scaffold.

Embodiment P29. The method of Embodiment 28, wherein said scaffold degrades at a rate of at least 1% per day during (b).

Embodiment P30. The method of any one of Embodiments 27-29, wherein said cells comprise stem cells or mature cells.

Embodiment P31. The method of any one of Embodiments 27-30, further comprising culturing said cells.

Embodiment P32. The method of any one of Embodiments 27-31, further comprising subjecting said cells to one or more expansion processes to expand said cells.

Embodiment P33. The method of Embodiment 32, wherein said scaffold is configured to facilitate cell expansion during said one or more expansion processes in a bioreactor chamber.

Embodiment P34. The method of any one of Embodiments 27-33, wherein (b) comprises generating differentiated or transdifferentiated cells from said cells, and optionally fusion of said differentiated or transdifferentiated cells within said scaffold.

Embodiment P35. The method of any one of Embodiments 27-34, wherein (a) comprises depositing at least a subset of said cells on a surface of the scaffold.

Embodiment P36. The method of Embodiment 35, wherein said surface is an adherent surface.

Embodiment P37. The method of any one of Embodiments 34-36, further comprising releasing cells of said at least said subset of said cells from said scaffold, and depositing said released cells on a surface of a separate scaffold.

Embodiment P38. The method of Embodiment 37, wherein said releasing is prior to (c).

Embodiment P39. The method of Embodiment 38, wherein, at least 50% of fusion of said differentiated or transdifferentiated cells occurs prior to said releasing.

Embodiment P40. The method of any one of Embodiments 31-39, wherein said culturing is conducted in the presence of said scaffold.

Embodiment P41. The method of any one of Embodiments 32-40, wherein said one or more expansion processes is conducted in the presence of said scaffold.

Embodiment P42. The method of any one of Embodiments 32-41, wherein said culturing and said one or more expansion processes are performed in a same bioreactor chamber.

Embodiment P43. The method of any one of Embodiments 32-42, wherein said culturing is performed in a bioreactor chamber and said one or more expansion processes are performed in an additional bioreactor chamber.

Embodiment P44. The method of Embodiment 43, wherein said additional bioreactor chamber comprises a plurality of additional bioreactor chambers each configured to facilitate an individual cell expansion process.

Embodiment P45. The method of Embodiment 43 or 44, further comprising directing at least a subset of cultured cells from said bioreactor chamber to said plurality of additional bioreactor chambers to perform a plurality of expansion processes.

Embodiment P46. The method of Embodiment 45, wherein expansion processes of said plurality of expansion processes are performed sequentially, simultaneously, or a combination thereof.

Embodiment P47. The method of Embodiment 45 or 46, wherein said plurality of additional bioreactor chambers comprises at least two bioreactor chambers.

Embodiment P48. The method of Embodiment 47, further comprising directing a medium through said bioreactor chamber and an additional bioreactor chamber of said plurality of additional bioreactor chambers to facilitate said culturing or said one or more expansion processes.

Embodiment P49. The method of Embodiment 48, wherein said medium is under continuous laminar flow.

Embodiment P50. The method of Embodiment 48 or 49, wherein said medium is configured to promote cell culturing or expansion processes.

Embodiment P51. The method of any one of Embodiments 48-50, further comprising directing said medium out of said additional bioreactor chamber.

Embodiment P52. The method of any one of Embodiments 48-51, further comprising filtering said medium directed out of said additional bioreactor chamber to remove undesired components from said medium, thereby generating a filtered medium.

Embodiment P53. The method of Embodiment 52, further comprising recycling said filtered medium into said bioreactor chamber.

Embodiment P54. The method of any one of Embodiments 27-53, wherein said cells comprise animal derived stem cells.

Embodiment P55. The method of any one of Embodiments 27-54, wherein said cells comprise porcine cells.

Embodiment P56. The method of any one of Embodiments 27-55, wherein said cells comprise pluripotent stem cells.

Embodiment P57. The method of any one of Embodiments 27-56, wherein said cells comprise embryonic stem cells (ESCs).

Embodiment P58. The method of Embodiments 27-57, wherein said cells comprise reprogrammed stem cells.

Embodiment P59. The method of any one of Embodiments 27-58, wherein said cells comprise induced pluripotent stem cells (iPSCs).

Embodiment P60. The method of any one of Embodiments 27-59, wherein said scaffold comprises a polymeric material.

Embodiment P61. The method of Embodiment 60, wherein said polymeric material comprises a synthetic polymeric material.

Embodiment P62. The method of Embodiment 61, wherein said synthetic polymeric material comprises a polyethylene glycol biomaterial.

Embodiment P63. The method of Embodiment 62, wherein said polyethylene glycol biomaterial comprises an arginylglycylaspartic (RGD) motif.

Embodiment P64. The method of any one of Embodiments 27-63, wherein said scaffold comprises a gellan gum biomaterial, a cassava biomaterial, a maize biomaterial, an alginate biomaterial, a corn-starch biomaterial, or any combination or variant thereof.

Embodiment P65. The method of any one of Embodiments 27-64, wherein said method is performed in vitro.

Embodiment P66. The method of any one of Embodiments 27-65, wherein said edible meat product is in a unit form of at least 50 grams.

Embodiment P67. The method of any one of Embodiments 27-66, wherein said edible meat product is in a solid state with a texture comparable with that of an in-vivo derived steak including loins.

Embodiment P68. The method of any one of Embodiments 27-66, wherein said edible meat product is in a solid state with a texture comparable with that of an in-vivo derived bacon.

Embodiment P69. The method of any one of Embodiments 27-66, wherein said edible meat product is in a solid state with a texture comparable with that of an in-vivo derived pork belly.

Embodiment P70. The method of any one of Embodiments 27-66, wherein said edible meat product is in a solid state with a texture comparable with that of an in-vivo derived mince.

Embodiment P71. The method of any one of Embodiments 27-66, wherein said edible meat product is in a solid state with a texture comparable with that of an in-vivo derived sausage.

Embodiment P72. The method of any one of Embodiments 27-66, wherein said edible meat product is in a solid state with a texture comparable with that of an in-vivo derived ribs.

Embodiment P73. The method of any one of Embodiments 27-66, wherein said edible meat product is in a solid state with a texture comparable with that of an in-vivo derived chops.

Embodiment P74. The method of any one of Embodiments 27-66, wherein said edible meat product is in a solid state with a texture comparable with that of an in-vivo derived cured meat product.

Embodiment P75. The method of any one of Embodiments 27-74, wherein said edible meat product is incorporated into a further processed food product.

Embodiment P76. The method of any one of Embodiments 27-75, wherein said edible meat product comprises nutritional additives comprising vitamins and minerals.

Embodiment P77. The method of any one of Embodiments 32-76, wherein said one or more expansion processes comprise passaging at least a subset of cultured cells.

Embodiment P78. The method of Embodiment 77, wherein said passaging comprises passing an enzyme over said at least said subset of said cultured cells to detach said cells from a surface of said scaffold.

Embodiment P79. A method for generating an edible meat product from cells, the method comprising: (a) modulating expression of one or more genes in said cells in a transient and non-integrative manner using two or more ectopic differentiation factors to generate progenitor cells; (b) differentiating at least a subset of said progenitor cells to generate terminally differentiated cells; and (c) producing said edible meat product based at least partially on said terminally differentiated cells.

Embodiment P80. The method of Embodiment 79, further comprising subjecting one or more of said cells, said progenitor cells, and said terminally differentiated cells to a culturing and/or an expansion process

Embodiment P81. The method of Embodiment 80, wherein said culturing and said expansion processes are performed in a same, or different bioreactor chambers.

Embodiment P82. The method of any one of Embodiments 79-81, wherein said terminally differentiated cells comprise muscle cells, fat cells, somite cells, neural cells, endothelial cells, smooth muscle cells, bone cells, or a combination thereof.

Embodiment P83. The method of any one of Embodiments 79-82, wherein said ectopic differentiation factors comprise nucleic acids, polypeptides, small molecules, growth factors, or any combination thereof.

Embodiment P84. The method of any one of Embodiments 79-83, wherein (b) comprises differentiating said progenitor cells by arresting the cell cycle of cells.

Embodiment P85. The method of any one of Embodiments 79-84, wherein said ectopic differentiation factors arrest the cell cycle of cells through reducing or removing growth factors from said cells.

Embodiment P86. The method of any one of Embodiments 79-85, wherein said growth factors comprise LIF, FGF, BMP, activin, MAPK, TGF-β, or any combination thereof.

Embodiment P87. The method of any one of Embodiments 79-86, wherein said arresting the cell cycle of cells occurs by reducing or removing serum levels in a solution in which cell culturing is conducted.

Embodiment P88. A method for generating an edible meat product using cells, the method comprising: (a) delivering into said cells two or more different types of nucleic acid molecules comprising messenger ribonucleic acid (mRNA), microRNA (miRNA), transfer RNA (tRNA), silencing RNA (siRNA), or complementary deoxyribonucleic acid (cDNA); (b) modulating gene expression of said cells with aid of said two or more different types of nucleic acid molecules or expression products thereof, to generate one or more target cells following delivery of said two or more different types of nucleic acid molecules, wherein said modulating is in a transient manner such that said nucleic acid molecules are not integrated into a genome of said cells; (c) producing said edible meat product using at least partially said one or more target cells generated in (b).

Embodiment P89. The method of Embodiment 88, wherein said two or more different types of nucleic acid molecules are generated by an in vitro process.

Embodiment P90. The method of Embodiment 88 or 89, wherein said two or more different types of nucleic acid molecules comprise mRNA and siRNA.

Embodiment P91. The method of Embodiment 90, wherein said mRNA comprises MYOD1, MYOG, MYF5, MYF6, PAX3, PAX7, or any combination or variant thereof.

Embodiment P92. The method of Embodiment 90 or 91, wherein said siRNA targets POUF51 (OCT3/4), KLF4, SOX2, or any combination or variant thereof.

Embodiment P93. The method of any one of Embodiment 88-92, wherein said two or more different types of nucleic acid molecules comprise cDNA and siRNA.

Embodiment P94. The method of Embodiment 93, wherein said cDNA comprises MYOD1, MYOG, MYF5, MYF6, PAX3, PAX7, or any combination or variant thereof.

Embodiment P95. The method of any one of Embodiments 88-94, wherein (b) comprises enhancing, reducing, or inhibiting said gene expression.

Embodiment P96. The method of any one of Embodiments 88-95, wherein said gene expression comprises expression of one or more genes in said cells.

Embodiment P97. The method of Embodiment 96, wherein (b) comprises enhancing expression of a first gene of said one or more genes, and inhibiting expression of a second gene of said one or more genes.

Embodiment P98. The method of Embodiment 97, wherein said delivering comprises a single dose of said two or more different types of nucleic acid molecules.

Embodiment P99. The method of Embodiment 97, wherein said delivering comprises at least two doses of said two or more different types of nucleic acid molecules.

Embodiment P100. The method of Embodiment 99, wherein individual doses of said at least two doses comprises different nucleic acid molecules.

Embodiment P101. The method of Embodiment 99 or 100, wherein said at least two doses comprise different concentrations of said two or more different types of nucleic acid molecules.

Embodiment P102. An edible meat product prepared by a process comprising the steps of: (a) bringing a plurality of cells in contact with a scaffold; (b) subjecting at least a subset of said plurality of cells to a differentiation or a transdifferentiation process in the presence of said scaffold and with the use of a growth factor or a nucleic acid molecule, to thereby generate a tissue; and (c) producing said edible meat product using said tissue.

Embodiment P103. The edible meat product of Embodiment 102, wherein said tissue comprises at least two types of cells.

Embodiment P104. The edible meat product of Embodiment 103, wherein said at least two types of cells comprise myocytes and adipocytes.

Embodiment P105. The edible meat product of Embodiment 104, wherein a ratio of said myocytes to said adipocytes is between 99:1 and 80:20.

Embodiment P106. The edible meat product of any one of Embodiments 102-105, wherein said edible meat product comprises at least 2% by mass of said scaffold.

Embodiment P107. The edible meat product of any one of Embodiments 102-106, wherein said edible meat product comprises less than 5% of muscle extracellular matrix by mass.

Embodiment P108. The edible meat product of any one of Embodiments 102-107, wherein said plurality of cells comprise stem cells or mature cells.

Embodiment P109. The edible meat product of any one of Embodiments 102-108, wherein said process further comprises culturing at least a subset of said plurality of cells.

Embodiment P110. The edible meat product of any one of Embodiments 102-109, wherein said process further comprises subjecting at least a subset of said plurality of cells to one or more expansion process.

Embodiment P111. The edible meat product of any one of Embodiments 102-110, wherein said scaffold comprises an extended 3-dimensional structure.

Embodiment P112. The edible meat product of any one of Embodiments 102-111, wherein (b) comprises generating differentiated or transdifferentiated cells from said cells, and optionally fusion of said differentiated or transdifferentiated cells within said scaffold.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-50. (canceled)

51. A method for differentiating or transdifferentiating cells to produce an edible meat product, the method comprising:
(a) delivering nucleic acid molecules comprising one or more ribonucleic acid (RNA) molecules into said cells;
(b) modulating gene expression of said cells with aid of said nucleic acid molecules or expression products thereof, to differentiate or transdifferentiate at least a subset of said cells to generate one or more target cells following delivery of said nucleic acid molecules, wherein upon said modulating, said nucleic acid molecules are not integrated into a genome of said cells; and
(c) producing said edible meat product using at least partially said one or more target cells generated in (b).

52. The method of claim 51, wherein said nucleic acid molecules comprise two or more different RNA molecules.

53. The method of claim 51, wherein said cells comprise animal derived stem cells.

54. The method of claim 51, wherein said cells comprise porcine cells.

55. The method of claim 51, wherein (c) comprises producing a tissue from said one or more target cells.

56. The method of claim 51, wherein said one or more target cells comprise at least two different types of cells.

57. The method of claim 56, further comprising co-culturing said at least two different types of cells to generate a three-dimensional tissue.

58. The method of claim 51, wherein said one or more RNA molecules encode MYOD1, MYOG, MYF5, MYF6, PAX3, or PAX7, or any combination or variant thereof.

59. The method of claim 51, wherein at least one of said RNA molecules is chemically modified to improve its stability.

60. The method of claim 51, wherein said nucleic acid molecules are delivered to said cells with neutral or anionic liposomes, cationic liposomes, lipid nanoparticles, ionizable lipids, or any combination or variation thereof.

61. The method of claim 51, wherein said nucleic acid molecules are delivered in a single dose to said cells.

62. The method of claim 51, wherein said nucleic acid molecules are delivered in at least two doses to said cells.

63. The method of claim 62, wherein individual doses of said at least two doses are delivered at least 3 days apart.

64. The method of claim 51, wherein said nucleic acid molecules are delivered at a concentration of at most 500 nM.

65. The method of claim 51, wherein said nucleic acid molecules comprise silencing RNA (siRNA) targeting POUF51 (OCT3/4), KLF4, SOX2, or any combination or variant thereof.

66. The method of claim 52, wherein said two or more different RNA molecules comprise messenger ribonucleic acid (mRNA), microRNA (miRNA), transfer RNA (tRNA), or silencing RNA (siRNA).

67. The method of claim 66, wherein said two or more different RNA molecules comprise said mRNA and said siRNA.

68. The method of claim 67, wherein said mRNA encodes MYOD1, MYOG, MYF5, MYF6, PAX3, PAX7, or any combination or variant thereof.

69. The method of claim 67, wherein said siRNA targets POUF51 (OCT3/4), KLF4, SOX2, or any combination or variant thereof.

70. The method of claim 67, wherein said delivering comprises delivering into said cell said two or more different RNA molecules in a single dose.
